SEQUENCE LISTING (11 sequences)

SEQ ID NO: 1 - protein, 51 aa, Artificial Sequence
  synthetic construct; positively charged carrier
  misc (1)..(20): the Gly at each of positions 1-20 can be present or absent
  misc (32)..(51): the Gly at each of positions 32-51 can be present or absent
GGGGGGGGGGGGGGGGGGGGRGRDDRRQRRRGGGGGGGGGGGGGGGGGGGG

SEQ ID NO: 2 - protein, 51 aa, Artificial Sequence
  synthetic construct; positively charged carrier
  misc (1)..(20): the Gly at each of positions 1-20 can be present or absent
  misc (32)..(51): the Gly at each of positions 32-51 can be present or absent
GGGGGGGGGGGGGGGGGGGGYGRKKRRQRRRGGGGGGGGGGGGGGGGGGGG

SEQ ID NO: 3 - protein, 49 aa, Artificial Sequence
  synthetic construct; positively charged carrier
  misc (1)..(20): the Gly can at positions 1-20 can be present or absent
  misc (30)..(49): the Gly can at positions 1-20 can be present or absent
GGGGGGGGGGGGGGGGGGGGRKKRRQRRRGGGGGGGGGGGGGGGGGGGG

SEQ ID NO: 4 - protein, 35 aa, Artificial Sequence
  synthetic construct; positively charged peptide
RKKRRQRRRGKKKKKKKKKKKKKKKGRKKRRQRRR

SEQ ID NO: 5 - protein, 45 aa, Artificial Sequence
  synthetic construct; positively charged peptide
  misc (1)..(20): Gly at positions 1-20 can be present or absent
  misc (26)..(45): Arg at positions 26-45 can be present or absent
GGGGGGGGGGGGGGGGGGGGRRRRRRRRRRRRRRRRRRRRRRRRR

SEQ ID NO: 6 - protein, 19 aa, Artificial Sequence
  synthetic construct; protein transduction domain
SGRQIKIWFQNRRMKWKKC

SEQ ID NO: 7 - protein, 39 aa, Artificial Sequence
  synthetic construct; positively charged peptide
YGRKKRRQRRRGKKKKKKKKKKKKKKKGYGRKKRRQRRR

SEQ ID NO: 8 - protein, 39 aa, Artificial Sequence
  synthetic construct; positively charged peptide
RGRDDRRQRRRGKKKKKKKKKKKKKKKGRGRDDRRQRRR

SEQ ID NO: 9 - protein, 20 aa, Artificial Sequence
  synthetic construct; poly-lysine
  misc (11)..(20): lys at positions 11-20 can be present or absent
LLLLLLLLLLLLLLLLLLLL

SEQ ID NO: 10 - protein, 10 aa, Artificial Sequence
  synthetic construct; protein transduction domain
GGGRRRRRRR

SEQ ID NO: 11 - protein, 84 aa, Artificial Sequence
  synthetic construct; positively charged peptide
  misc (10)..(17): the Gly at positions 10-17 can be present or absent
  misc (23)..(47): the Lys at positions 23-47 can be present or absent
  misc (48)..(55): the Gly at positions 48-55 can be present or absent
RKKRRQRRRGGGGGGGGKKKKKKKKKKKKKKKKKKKKKKKKKKKKKKKKKKKKKKKKKKKKKKKKKKGGGGGGGGRKKRRQRRR

(19) United States
(12) Patent Application Publication
(10) Pub. No.: US 2023/0210963 A1
(43) Pub. Date: Jul. 6, 2023

(54) INJECTABLE BOTULINUM TOXIN METHOS FOR TREATING HEADACHES

(71) Applicant: Revance Therapeutics, Inc., Newark, CA (US)

(72) Inventors: Mahan Chehrenama, Great Falls, VA (US); Stephen Silberstein, Philadelphia, PA (US); Rami Burstein, Boston, MA (US); Peter McAllister, Rowayton, CT (US); Paul Matthew, Braintree, MA (US); Andrew Blumenfeld, Del Mar, CA (US); Jessica Ailani, McClean, VA (US); David Dodick, Scottsdale, AZ (US); Roman Rubio, Redwood City, CA (US)

(21) Appl. No.: 17/912,513

(22) PCT Filed: Mar. 18, 2021
(86) PCT No.: PCT/US2021/023046

§ 371 Date: Sep. 18, 2022

Related U.S. Application Data
(60) Provisional application No. 62/991,185, filed on Mar. 18, 2020.

(51) Int. Cl. A61K 38/48; A61K 9/00; A61P 25/06; A61K 39/395

(52) U.S. Cl. CPC A61K 38/4893; A61K 9/0019; A61K 39/3955; A61P 25/06

(57) ABSTRACT

Provided is an invention based on methods to treat or prevent headaches with injectable compositions comprising botulinum toxin that may be administered using an injection paradigm that provides botulinum toxin at one or more locations of neuronal activity associated with EEG detectable brain cortical electrical activity to a subject suffering from such malady. Co-therapuetics therewith are also disclosed.

### CROSS REFERENCE TO RELATED APPLICATIONS

This application claims the benefit of of U.S. Provisional Application No. 62/991,185, filed Mar. 18, 2020, which is incorporated by reference in its entirety.

### FIELD OF THE INVENTION

The methods described herein generally relate to the fields of medical treatments involving botulinum toxin. More specifically, this invention relates to methods to treat or prevent headaches, including migraines, or a disorder related thereto, or a symptom thereof, with injectable compositions comprising botulinum toxin that may be administered using an injection paradigm that provides botulinum toxin at one or more locations of neuronal activity associated with EEG detectable brain cortical electrical activity to a subject suffering from such maledy.

### BACKGROUND

Head pain can be classified as being one of three types: a) primary headache, b) secondary headache, and c) cranial neuralgias, facial pain, and other headaches. Common primary headaches include tension-type, migraine, and trigeminal autonomic cephalalgias (for example, cluster headaches). Common secondary headaches include medication-overuse headaches, headaches attributed to trauma or injury to the head and/or neck, and headaches attributed to cranial or cervical vascular disorders. Medication overuse headache (rebound headache) is a condition where frequent use of pain medications can lead to persistent head pain or worsening of a pre-existing headache phenotype. Closed head trauma is the most common form of traumatic brain injury and mild traumatic brain injury is the most common form of any traumatic brain injury. Post-traumatic headache is usually the earliest symptom after traumatic brain injury and also one of the signature persistent residual symptoms. Additionally, headache is also frequently seen as an adverse event after craniotomy.

Migraine is a neurological condition that can cause multiple symptoms and is frequently characterized by intense, debilitating headaches. The disorder has a profoundly negative impact on the quality of life of millions of people. Migraine is the second global cause of years lived with disability. It is estimated that in the United States alone, over 36 million people suffer from migraine disease, and the condition affects about 1 billion people worldwide.

Different subtypes of migraine exist, for example migraine without aura, migraine with aura, migraine with brainstem aura, migraine without head pain (“acephalgic migraine”), hemiplegic migraine, retinal migraine, and chronic migraine.. Chronic migraine (CM) and episodic migraine (EM) are part of the spectrum of migraine disorders, but they are distinct clinical entities. For example, frequency of headache days and degree of disability distinguish CM from EM.

CM is defined as headache occurring at least 15 days per month, with at least 8 of those days meeting criteria for migraine (light sensitivity and noise sensitivity, or nausea must be present), in this pattern, for more than 3 months. Symptoms may include nausea, vomiting, difficulty speaking, numbness or tingling, and sensitivity to light and sound.

EM is defined as less than 15 headache days per month. The prevalence of EM (12-15%) is higher than CM (2-5%); however, exact data for EM subtypes (low-frequency and high-frequency), as available for CM, are not available. Different studies have used frequencies from 8 to 14 and 10 to 14 migraine headache days per month to define low-frequency EM (LFEM) and high-frequency EM (HFEM). EM progresses to CM at the rate of 2.5% per year and CM often remits to EM (2-year transition rate of 26%). Medication overuse is the most common reason why EM turns chronic, although there are many other variables that may contribute to migraines becoming chronic.

While medications for abortive treatment to reduce the pain and additional symptoms, and medications for preventive treatment to reduce the frequency and severity are treatment options, a significant number of patients discontinue medication due to ineffectiveness and/or side effects. Examples of medications for abortive treatment include nonsteroidal anti-inflammatory drugs (e.g. diclofenac, aspirin, acetaminophen, ketorolac), ergotamines (e.g. ergotamine (Ergomar), ergotamine and caffeine (Cafatine, Cafergot, Cafetrate, Ercaf, Migranol, Migergot, Wigraine), methysergide, methylergonovine (Methergine)), triptans (e.g. Sumatriptan, Frovatriptan, Rizatriptan, Sivatriptan), anti-nausea drugs (e.g. dimenhydrinate (Dramamine, metoclopramide (Reglan), prochlorperazine (Compazine)), and opioids (e.g. codeine, morphine, oxycodone). Examples of medications for preventive treatment include CGRP antagonists (e.g. erenumab (Aimovig), fremanezumab (Ajovy)), beta-blockers (e.g. atenolol (Temormin), metoprolol (Toprol XL), propranolol (Corgard)), calcium-channel blockers (e.g. diltiazem (Cardizem, Cartia XT, Dilacor, Tiazac), nimodipine (Nimotop), verapamil (Calan, Covera, Isoptin, Verelan)), antidepressants (e.g. amitriptyline (Elavil, Endep), fluoxetine (Prozac, Sarafem), imipramine (Tofranil)), and anticonvulsants (e.g. gabapentin (Neurontin), levetiracetam (Keppra), pregabalin (Lyrica)). Two thirds of migraine sufferers delay or avoid taking current prescription medication because of treatment side effects. Common side effects of most of these drugs include nausea, severe constipation, sleepiness, fatigue, trouble functioning, and difficulty thinking.

In 2011, the FDA approved botulinum toxin (Botox®) injections in the forehead and neck muscles for the treatment of CM. However, Botox® treatment, commonly by injection, has a number of disadvantages.

Botulinum toxins (also known as botulin toxins or botulinum neurotoxins) are neurotoxins produced by the gram-positive bacteria Clostridium botulinum. They act to produce paralysis of muscles by preventing synaptic transmission by inhibiting the release of acetylcholine across the neuromuscular junction and are thought to act in other ways as well. They essentially block signals that normally cause muscle spasms or contractions, resulting in paralysis. In addition, observations suggest that the mode of action of botulinum toxin in migraine may not be limited to the injection site, but also include anatomically connected sites due to axonal transport. The mechanism of action behind the effect of botulinum toxin in migraine may also include modulation of neurotransmitter release (for example acetylcholine), changes in surface expression of receptors and cytokines, as well as opiodergic transmission.

Botulinum toxin is classified into eight serologically related but distinct neurotoxins. Of these, seven can cause paralysis, namely botulinum neurotoxin serotypes A, B, C, D, E, F and G. Each serotype is distinguished by neutralization with type-specific antibodies. Nonetheless, the molecular weight of the neuroactive botulinum toxin protein molecule, for all seven of these active botulinum toxin serotypes, is about 150 kDa. As released by the bacterium, the botulinum toxins are complexes comprising the 150 kDa botulinum toxin protein molecule associated with other non-toxin proteins. The botulinum toxin type A complex can be produced by the Clostridia bacterium as 900 kDa, 500 kDa, and 300 kDa forms. Botulinum toxin types B and C are apparently produced as only a 700 kDa or 500 kDa complex. Botulinum toxin type D is produced as both 300 kDa and 500 kDa complexes. Botulinum toxin types E and F are produced as approximately 300 kDa complexes. The complexes having molecular weights greater than about 150 kDa are believed to contain a non-toxin hemagglutinin protein and a non-toxin and non-toxic hemagglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. In addition, it is possible that the larger (greater than about 150 kDa molecular weight) botulinum toxin complexes result in a slower rate of diffusion of the botulinum toxin away from a site of intramuscular injection of a botulinum toxin complex.

The different serotypes of botulinum toxin vary in the animal species they affect and in the severity and duration of paralysis they evoke. For example, it has been determined that botulinum toxin type A is 500 times more potent, as measured by the rate of paralysis produced in the rat, than is botulinum toxin type B. In addition, botulinum toxin type B has been determined to be non-toxic in primates at a dose of 480 U/kg, about 12 times the primate LD50 for type A. Due to the molecular size and structure of botulinum toxin, it cannot cross stratum corneum and the multiple layers of the underlying skin architecture.

Despite the potent toxicity of botulinum toxin of subtype A, the muscle-paralyzing effects of botulinum toxin have been used for both cosmetic and therapeutic effects. Controlled administration of botulinum toxin, typically by injection, has been used to provide muscle paralysis to treat conditions, for example, neuromuscular disorders characterized by hyperactive skeletal muscles. Conditions that have been treated with botulinum toxin include hemifacial spasm, adult onset spasmodic torticollis, anal fissure, blepharospasm, cerebral palsy, cervical dystonia, migraine headaches, strabismus, temporomandibular j oint disorder, and various types of muscle cramping and spasms. More recently, the muscle-paralyzing effects of botulinum toxin have been advantageous in cosmetic applications, such as treatment of wrinkles, frown lines, as well as other conditions caused by spasms or contractions of facial muscles.

To provide additional stability to botulinum toxin, the toxin complexes are conventionally stabilized by combining the complexes with albumin during manufacturing. For example, BOTOX® (Allergan, Inc., Irvine, CA) is a botulinum toxin-containing formulation that contains 100 U of type A botulinum toxin with accessory proteins, 0.5 milligrams of human albumin, and 0.9 milligrams of sodium chloride. The albumin serves to bind and to stabilize toxin complexes in disparate environments, including those associated with manufacturing, transportation, storage, and administration.

Formulations that do not include any animal-based proteins, including albumin, have the advantage of avoidance of potential antigenic or other complications that may be associated with purified or recombinantly produced proteins. Such animal-free botulinum toxin formulations are disclosed in, for example, U.S. Pat. Nos. 9,956,435, 10,111,939, and 9,198,958, and US2009/0324647, each hereby incorporated herein by reference in its entirety.

Typically, the botulinum toxin is administered to patients by carefully controlled injections of compositions containing botulinum toxin complex and albumin. However, there are several problems associated with this approach. Not only are the injections painful, but typically large subdermal wells of toxin are locally generated around the injection sites, in order to achieve the desired therapeutic or cosmetic effect. The botulinum toxin may migrate from these subdermal wells to cause unwanted paralysis in surrounding areas of the body. This problem is exacerbated when the area to be treated is large and many injections of toxin are required to treat the area. Moreover, because the injected toxin complexes contain non-toxin proteins and albumin that stabilize the botulinum toxin and increase the molecular weight of the toxin complex, the toxin complexes have a long half-life in the body and may cause an undesirable antigenic response in the patient. For example, some patients will, over time, develop an allergy to the albumin used as a stabilizer in current commercial formulations. Also, the toxin complexes may induce the immune system of the patient to form neutralizing antibodies, so that larger amounts of toxin are required in subsequent administrations to achieve the same effect. When this happens, subsequent injections must be carefully placed so that they do not release a large amount of toxin into the bloodstream of the patient, which could lead to fatal systemic poisoning, especially since the non-toxin proteins and albumin stabilize the botulinum toxin in blood.

Treatments for migraine headaches include oral medications, botulinum toxin injections, subcutaneous and intravenous injections, as well as alternative and complemantary therapies. The use of botulinum toxin, typically type A (although Type B has also been used) can prevent headaches in patients with CM. Various protocols for using botulinum toxin to treat headaches have been described in, for example, U.S. Pat. Nos. 9,504,735, 7,655,244, 10,092,631, and 9,078,893. The FDA-approved protocol PREEMPT involves 31 injections (155 Units) in seven key areas of the head and neck: Frontalis, corrugator, procerus, occipitalis, temporalis, trapezius, and cervical paraspinal muscle group. Doctors injecting the toxin may select additional muscles and areas to be injected in which patients particularly have pain in addition to fixed sites, a treatment strategy called “follow the pain” [ https://www.botoxmedical.com/Common/Assets/APC55BL15%20Injection%20Workbook %20FINAL%20elec.pdf]. Each muscle and/or area affected by migraine typically has to be injected separately. As such, based on the diffusion characteristics of currently available toxin formulations, there is a limit to the total quantity of toxin that can be injected into the body at one time. While the treatment for migraine headaches involves regular intervention, which takes effect over a period of 4-7 days or longer after injection, the response to the treatment with botulinum toxin typically wears off a few weeks, often as early as week 10 before re-injection is scheduled at week 12, requiring the person suffering from migraine disease to be injected again.

### SUMMARY OF THE INVENTION

In one of its aspects, the invention relates to a method for treating or reducing the frequency and/or severity of headaches by administering by injection a dose of an injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the total treatment dose of botulinum toxin component administered to the individual is 100 U to 450 U which is administered in one or more injections sites.

In some embodiments, the headache is selected from migraine headache, a post-traumatic headache, a post-craniotomy headache, tension-type headache, cluster headache, and/or medication-overuse headache. In preferred embodiments, the migraine headache is a chronic or an episodic migraine headache. For instance, in some embodiments, the migraine headache is an episodic migraine headache. In some embodiments, the headache is a high-frequency episodic migraine headache. In some embodiments, the migraine headache is a chronic migraine headache.

In some embodiments, the invention relates to a method for treating or reducing the frequency and/or severity of headaches by administering by injection a dose of an injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the composition further comprises a positively charged carrier comprising a positively charged backbone and at least one efficiency group; and wherein the total treatment dose of botulinum toxin component administered to the individual is 100 U to 450 U which is administered in one or more injections sites.

In an embodiment, the botulinum toxin is present in the composition in a total dosage amount from more than 100 U, 100 to 200 U, 200 to 300 U, or 300-450 U. In an embodiment, the botulinum toxin is present in the composition in a dosage amount selected from the group consisting of 100 U, 200 U, 300 U and 450 U. In certain embodiments, the duration of the treatment effect (also referred to as the “efficacy window”) can be greater than 2 months; greater than 3 months; greater than 4 months; greater than 5 months; greater than 6 months; or at least 6 months through 10 months. In preferred embodiments the 150 kDa type A botulinum toxin form is used at these dosage amounts. In preferred embodiments daxibotulinumtoxin A is used at these dosage amounts.

In some embodiments, the positively charged backbone is polylysine or polyethyleneimine. In some embodiments, the positively charged backbone is a polylysine backbone wherein one or more positively charged efficiency groups are attached to said polylysine backbone. In some embodiments, the positively charged efficiency groups are either protected oligoarginine or TAT or modified TAT domains. In some embodiments, the positively charged efficiency groups are selected from an amino acid sequence selected from the group consisting of (gly)ₚ-RGRDDRRQRRR-(gly)_{q} (SEQ ID NO: 1), (gly)ₚ-YGRKKRRQRRR-(gly)_{q} (SEQ ID NO: 2), and (gly)ₚ-RKKRRQRRR-(gly)_{q} (SEQ ID NO: 3), wherein the subscripts p and q are each independently an integer of from 0 to 20 and mixtures thereof. In preferred embodiments, the positively charged efficiency groups include the amino acid sequence RKKRRQRRR(gly)_{q}-(K)ₓ-(gly)ₚ-RKKRRQRRR, wherein the subscript x is an integer from 5 to 50 or 10-30 or 10 to 20, wherein p and q are each an integer from 0 to 8. For instance, in a preferred embodiment, the positively charged efficiency group has the amino acid sequence RKKRRQRRRG-(K)₁₅-GRKKRRQRRR (SEQ ID NO: 4).

In a preferred embodiment, the invention relates to a method for treating or reducing the frequency and/or the severity of headaches by administering by injection a dose of a sterile injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the composition further comprises a positively charged carrier comprising a positively charged backbone and at least one efficiency group; wherein the positively charged efficiency group has the amino acid sequence RKKRRQRRRG-(K)₁₅-GRKKRRQRRR (SEQ ID NO: 4); wherein the total treatment dose of botulinum toxin component administered to the individual is 100 U to 450 U which is administered in one or more injections sites; and wherein the duration of the treatment effect comprises greater than 2 months; greater than 3 months; greater than 4 months; greater than 5 months; or at least 6 months through 10 months.

In one of its aspects, the invention relates to a method for treating or reducing the frequency and/or severity of headaches by administering by injection a dose of a sterile injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the injection sites correspond to one or more electrode placement sites selected from Fpz, Fp1, Fp2, F3, F4, F7, F8, T3, T4, C3, C4, A1, A2, T5, P3, P4, T6, O1, O2, Ground 1 (GND1), or Ground 2 (GND2) (Table 1). In some embodiments, the injection sites further include muscle injection sites selected from trapezius or masseter. In some embodiments, the injection sites further correspond to one or more electrode placement sites selected from Cz, Pz, or Oz.

TABLE. 1

Injection Sites with EEG Detectable Brain Cortical Electrical Activity

| | | | |
|---|---|---|---|
| Brain area | Left | Midline | Right |
| Frontal Pole (FP) | FP1 | Fpz | FP2 |
| Frontal / Forehead (Ground) | GND1 | | GND2 |
| Frontal (F) | F2 | | F2 |
| Inferior Frontal (F) | F7 | | F8 |
| Mid Temporal (T) | T7 | | T8 |
| Posterior Temporal (P) | P7 | | P8 |
| Central (C) | C3 | Cz | C4 |
| Parietal (P) | P3 | Pz | P4 |
| Occipital (O) | O1 | Oz | O2 |
| Mastoid/Ear/Auricular (A) | A1 | | A2 |

In some embodiments, the composition is administered at 10 to 25 injection sites, wherein 10 to 21 injection sites are EEG-associated injection sites and 2 to 4 injection sites are muscle injection sites; wherein the EEG-associated injection sites are selected from among Fpz, Fp1, Fp2, F3, F4, F7, F8, T3, T4, C3, C4, A1, A2, T5, P3, P4, T6, O1, O2, GND1, and GND2, and the muscle injection sites are selected from right and left trapezius muscle and right and left masseter muscle. In preferred embodiments, the composition is administered at 17 injection sites; wherein the injection sites are Fp1, Fp2, T3, T4, T5, T6, O1, O2, F3, F4, P3, P4, F7, F8, GND1, GND2, and Fpz. In preferred embodiments, the composition is administered at 17 EEG-associated injection sites and 2 muscle injection sites, wherein the 17 EEG-asscoiated injection sites are Fp1, Fp2, T3, T4, T5, T6, O1, O2, F3, F4, P3, P4, F7, F8, GND1, GND2, Fpz, and the 2 muscle injection sites are the right and left trapezius muscle. In yet other embodiments, the composition is administered at 17 EEG-associated injection sites and 3 muscle injection sites, wherein the 17 EEG-associated injection sites are Fp1, Fp2, T3, T4, T5, T6, O1, O2, F3, F4, P3, P4, F7, F8, GND1, GND2, Fpz, and the 3 muscle injection sites are the right and left trapezius muscle, and the right or left masseter muscle. In other embodiments, the composition is administered at 17 EEG-associated injection sites and 4 muscle injection sites, wherein the 17 EEG-associated injection sites are Fp1, Fp2, T3, T4, T5, T6, O1, O2, F3, F4, P3, P4, F7, F8, GND1, GND2, Fpz, and the 4 muscle injection sites are the right and left trapezius muscle and the right and left masseter muscle. In some embodiments, the injection sites further correspond to one or more electrode placement sites selected from Cz, Pz, or Oz. Identification of the injection location sites associated with EEG electrode placement sites, does not limit the practioner from making additional injections without departing from this invention and would be within the scope of the invention disclosed herein.

In a preferred embodiment, the invention relates to a method for treating or reducing the frequency and/or the severity of headaches by administering by injection a dose of a sterile injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the botulinum toxin is of serotype A; wherein the composition is administered at 10 to 25 injection sites, wherein 10 to 21 injection sites are EEG-associated injection sites and 2 to 4 injection sites are muscle injection sites; wherein the EEG-associated injection sites are selected from among Fpz, Fp1, Fp2, F3, F4, F7, F8, T3, T4, C3, C4, A1, A2, T5, P3, P4, T6, O1, O2, GND1, and GND2, and the muscle injection sites are selected from right and left trapezius muscle and right and left masseter muscle; wherein the total treatment dose of botulinum toxin component administered to the individual is 100 U to 450 U which is administered in one or more injections sites; and wherein the duration of the treatment effect comprises greater than 2 months; greater than 3 months; greater than 4 months; greater than 5 months; or at least 6 months through 10 months.

In a preferred embodiment, the invention relates to a method for treating or reducing the frequency and/or the severity of headaches by administering by injection a dose of a sterile injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the botulinum toxin is of serotype A; wherein the composition is administered to the injection sites consisting of Fpz, Fp1, Fp2, F3, F4, F7, F8, T3, T4, T5, P3, P4, T6, O1, O2, GND1, GND2, trapezius muscles, and optionally masseter muscles; wherein the total treatment dose of botulinum toxin component administered to the individual is 100 U to 450 U which is administered in one or more injections sites; and wherein the duration of the treatment effect comprises greater than 2 months; greater than 3 months; greater than 4 months; greater than 5 months; or at least 6 months through 10 months.

In a preferred embodiment, the invention relates to a method for treating or reducing the frequency and/or the severity of headaches by administering by injection a dose of a sterile injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the botulinum toxin is of serotype A; wherein the composition further comprises a positively charged carrier comprising a positively charged backbone and at least one efficiency group; wherein the positively charged efficiency group has the amino acid sequence RKKRRQRRRG-(K)₁₅-GRKKRRQRRR (SEQ ID NO: 4); wherein the composition is administered at 10 to 25 injection sites, wherein 10 to 21 injection sites are EEG-associated injection sites and 2 to 4 injection sites are muscle injection sites; wherein the EEG-associated injection sites are selected from among Fpz, Fp1, Fp2, F3, F4, F7, F8, T3, T4, C3, C4, A1, A2, T5, P3, P4, T6, O1, O2, GND1, and GND2, and the 2 to 4 muscle injection sites are selected from right and left trapezius muscle and right and left masseter muscle; wherein the total treatment dose of botulinum toxin component administered to the individual is 100 U to 450 U which is administered in one or more injections sites; and wherein the duration of the treatment effect comprises greater than 2 months; greater than 3 months; greater than 4 months; greater than 5 months; or at least 6 months through 10 months. In some embodiments, the injection sites further correspond to one or more electrode placement sites selected from Cz, Pz, or Oz.

In a preferred embodiment, the invention relates to a method for treating or reducing the frequency and/or the severity of headaches by administering by injection a dose of a sterile injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the botulinum toxin is of serotype A; wherein the composition further comprises a positively charged carrier comprising a positively charged backbone and at least one efficiency group; wherein the positively charged efficiency group has the amino acid sequence RKKRRQRRRG-(K)₁₅-GRKKRRQRRR (SEQ ID NO: 4); wherein the composition is administered to 17 EEG-associated injection sites and further 2 to 4 muscle injection sites, wherein the 17 EEG-associated injection sites are Fpz, Fp1, Fp2, F3, F4, F7, F8, T3, T4, T5, P3, P4, T6, O1, O2, GND1, GND2, and the 2 to 4 muscle injection sites are the right and left trapezius muscle without administration to the masseter muscle (2 muscle sites), the right and left trapezius muscle with unilateral administration of the masseter (3 muscle sites), or the right and left trapezius with bilateral administration of masseter (4 muscle sites); wherein the total treatment dose of botulinum toxin component administered to the individual is 100 U to 450 U which is administered in one or more injections sites; and wherein the duration of the treatment effect comprises greater than 2 months; greater than 3 months; greater than 4 months; greater than 5 months; or at least 6 months through 10 months.

In some embodiments, the total treatment dose of botulinum toxin component administered to the individual is 100 U to 450 U is administered in one or more injections sites. In some embodiments, the total treatment dose of botulinum toxin component is administered to the individual is 100 to 200 U, 200 U to 300 U, or 300 U to 450 U, and 376 U, which is administered in one or more injections sites.

Modified injection techniques as described herein involve administration to allow for maximizing the dose while minimizing the number of injections required per site and thus maximizing the effect on cortical regions; while minimizing the side effects such as pain, swelling, muscle weakness, muscle paralysis, and muscle atrophy. In some embodiments, the treatment dose of botulinum toxin is administered using one or more injection techniques of interest, wherein the injection technique can be sleeving, angling, tunneling, single injection, or serial injection, or a modification thereof. In some embodiments, the composition is injected at T5, T6, O1, and O2; wherein the injection technique used is sleeving. In some embodiments, the composition is injected at T5, T6, O1, O2, FP1, FP2, F3, F4, P3, and P4; wherein the injection technique is sleeving. In some embodiments, the composition is injected at T3, T4, and in the trapezius muscle; wherein the injection technique is angling.

The methods described herein can deliver botulinum toxin in an effective amount for treating or preventing migraine disease and headaches generally. In some embodiments, the compositions are administered in a volume of about 100 µL to about 400 µL, about 100 µL to about 200 µL, about 200 µL to about 300 µL, about 300 µL to about 400 µL, about 100 µL; about 200 µL; about 300 µL, or about 400 µL; in preferred embodiments the injection volume is 100 µL, 200 µL or 400 µL.

In a further aspect, the invention relates to a combination therapy involving the administration of the injectable botulinum toxin composition as described herein in addition to one or more co-therapeutics. The one or more co-therapeutics can be administered seperately, sequentially, or simultaneously with the injectable botulinum toxin composition, including in some embodiments, administration of the botulinum toxin composition together with the one or more co-therapeutics as a single injectable formulation. In some embodiments, the one or more co-therapeutics are administered near the end of the efficacy window of botulinum toxin, such as at about 1, 2, 3, or 4 weeks before the end of the treatment window. Efficacy window for botulinum toxin (i.e., the duratiton of action) can be from 12 weeks to 24 weeks, such as about 14 weeks, 16 weeks, 18 weeks, 20 weeks, 22 weeks, or 24 weeks. Thus, the one or more co-therapeutics can be administered at 10, 11, 12, 13, 14, 15, 16, 17, 18, 19, 20, 21, or 22 weeks after a treatment with botulinum toxin.

Suitable co-therapeutics can be selected from an antagonist of calcitonin gene-related peptide (CGRP-antagonist), a 5-HT-1F receptor agonist, and an ergot compound. Suitable CGRP-antagonists include both antibodies (and antigen-binding fragments thereof) and small molecule CGRP-antagonists. In some embodiments, the CGRP-antagonist is an anti-CGRP antibody or an antigen binding fragment thereof. Non-limiting examples include galcanezumab, fremanezumab, eptinezumab, erenumab, and combinations thereof. Anti-CGRP antibodies or antigen binding fragments thereof can be administered with the botulinum toxin composition seperately, sequentially, or simultaneously. In some embodiments, the anti-CGRP antibody can be administered together with the botulinum toxin composition as a single injection at a suitable injection site as described further herein. In other embodiments, the anti-CGRP antibody can be administered seperately from or sequentially with the botulinum toxin composition according to known methods of administering such antibodies.

In further embodiments, the CGRP-antagonist is a gepant. Non-limiting examples of suitable gepants include ubrogepant, rimegepant, atogepant, vazegepant, combinations thereof, and pharmaceutically-acceptable salts thereof. Such small molecule CGRP-antagonists can be administered seperately, sequentially, or simultaneously with the injectable botulinum toxin composition. In some embodiments, the gepant can be administered at a suitable oral dose 1-3 times per day to an individual that is concurrently undergoing treatment with the injectable botulinum toxin composition. In further embodiments, the gepant can be administered together with the botulinum toxin composition as a single injectable dose.

In some embodiments, the combination therapeutic methods can be useful for any individual suffering from headaches such as migraines. In a further embodiment, the individual receiving the combination therapy can be a non-responder or insufficient responder to one or more triptan drugs.

Another embodiment of the present disclosure is a combination therapy involving the administration of the botulinum toxin composition with both a CGRP-antagonist and a 5-HT-1F receptor agonist. The three therapeutic agents can be administered seperately, sequentially, or simultaneously, e.g., in some embodiments, in a single injection. In other embodiments, the individual in need of treatment can be administered the CGRP-antagonist and 5-HT-1F receptor agonist according to known routes of administration, seperately from the botulinum toxin composition. Non-limiting examples of suitable 5-HT-1F receptor agonists include without limitation a ditan or a pharmaceutically-acceptable salt thereof. The ditan, for example, can be lasmiditan or a pharmaceutically-acceptable salt thereof.

Similarly, another embodiment of the present disclosure is a a combination therapy involving the administration of the botulinum toxin composition with both a CGRP-antagonist and an ergot compound. Suitable ergot compounds include ergotamine tartrate, ergonovine maleate, and ergoloid mesylates. Specific, non-limiting examples include dihydroergocornine, dihydroergocristine, dihydroergocryptine, and dihydroergotamine mesylate (DHE 45).

### BRIEF DESCRIPTION OF THE DRAWINGS

FIG. 1 depicts electrode placement in the standard 10/20 System.

FIG. 2 shows standard 10/20 system method of EEG electrode placement.

FIG. 3 shows injection locations based on the FTE (“follow the electrode”) paradigm. Both frontal and rear/side views are shown. Each circle indicates an injection site. The size of the circle is representative of the botox distribution area achieved by use of special injection technique. Purple circles are mandatory injection sites, while pink circles are optional.

FIG. 4 shows exemplary drug administration plan for patients.

Other aspects, features and advantages of the invention will become apparent from the following detailed description and illustrative examples.

### DETAILED DESCRIPTION

This invention relates to a method for treating or reducing the frequency and/or severity of headaches by administering by injection a dose of a sterile injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition; wherein the botulinum toxin composition comprises a botulinum toxin component and a pharmaceutically acceptable diluent suitable for injection; and wherein the botulinum toxin component is selected from the group consisting of a botulinum toxin, a botulinum toxin complex, or a reduced botulinum toxin complex; wherein the total treatment dose of botulinum toxin component administered to the individual is 100 U to 450 U which is administered in one or more injections sites.

Use of reference to EEG locations is only to provide reference points for injection location and is not meant to suggest actual use of EEG with electrical recordings.

In some embodiments, the composition can afford beneficial reductions in immune responses to the botulinum toxin. In embodiments, the injectable compositions of the invention provide an effect lasting at least 2 months, at least 3 months, or greater than 3 months, for example, up to about 6 months, in subjects to whom such compositions, particularly those comprising botulinum toxin in amounts of 100 U or more, are administered by injection for the treatment or prevention of headaches.

### Botulinum Toxin

The term “botulinum toxin” as used herein may refer to any of the known types of botulinum toxin (e.g., 150 kD botulinum toxin protein molecules associated with the different serotypes of C. botulinum), whether produced by the bacterium or by recombinant techniques, as well as any types that may be subsequently discovered including newly discovered serotypes, and engineered variants or fusion proteins. As mentioned above, currently seven immunologically distinct botulinum neurotoxins have been characterized, namely botulinum neurotoxin serotypes A, B, C1, D, E, F and G, each of which is distinguished by neutralization with type-specific antibodies. The different serotypes of botulinum toxin vary in the animal species that they affect and in the severity and duration of the paralysis they evoke. In preferred embodiments, the composition comprises a botulinum toxin of serotype A.

The botulinum toxin serotypes are commercially available, for example, from Sigma-Aldrich (St. Louis, MO) and from Metabiologics, Inc. (Madison, WI), as well as from other sources. At least two types of botulinum toxin, types A and B, are available commercially in formulations for treatment of certain conditions. Type A, for example, is contained in preparations of Allergan, Inc., having the trademark BOTOX®, as well as in preparations of Ipsen Limited, having the trademark DYSPORT®. The original Botox® formulation, was prepared by Schantz in 1979 (Schantz et al., “Preparation and characterization of botulinum toxin type A for human treatment” Therapy with Botulinum Toxin. Vol. 109. New York, NY: Marcel Dekker; 1994. pp. 10-24). Type B is contained, for example, in preparations of Elan Pharmaceuticals having the trademark MYOBLOC®. Recombinant botulinum toxin can also be purchased, e.g., from List Biological Laboratories, Campbell, CA.

The term “botulinum toxin” used in the methods of this invention can alternatively refer to a botulinum toxin derivative, that is, a compound that has botulinum toxin activity but contains one or more chemical or functional alterations on any part or on any amino acid chain relative to naturally occurring or recombinant native botulinum toxins. For instance, the botulinum toxin may be a modified neurotoxin that is a neurotoxin which has at least one of its amino acids deleted, modified or replaced, as compared to a native form, or the modified neurotoxin can be a recombinantly produced neurotoxin or a derivative or fragment thereof. For instance, the botulinum toxin may be one that has been modified in a way that, for instance, enhances its properties or decreases undesirable side effects, but that still retains the desired botulinum toxin activity. Alternatively the botulinum toxin used in this invention may be a toxin prepared using recombinant or synthetic chemical techniques, e.g. a recombinant peptide, a fusion protein, or a hybrid neurotoxin, for example prepared from subunits or domains of different botulinum toxin serotypes (See, U.S. Pat. No. 6,444,209, for instance). The botulinum toxin may also be a portion of the overall molecule that has been shown to possess the necessary botulinum toxin activity, and in such case may be used per se or as part of a combination or conjugate molecule, for instance a fusion protein. Alternatively, the botulinum toxin may be in the form of a botulinum toxin precursor, which may itself be non-toxic, for instance a non-toxic zinc protease that becomes toxic on proteolytic cleavage.

The term “botulinum toxin complex,” or “toxin complex,” as used herein refers to the approximately 150 kD botulinum toxin protein molecule (belonging to any one of botulinum toxin serotypes A-G), along with associated endogenous non-toxin proteins (i.e., hemagglutinin protein and non-toxin non-hemagglutinin protein produced by C. botulinum bacteria). In some embodiments, the botulinum toxin complex need not be derived from C. botulinum bacteria as one unitary toxin complex, but rather may be, for example, botulinum toxin that is recombinantly prepared first and then subsequently combined with the non-toxin proteins.

The term “reduced botulinum toxin complex,” or “reduced toxin complex,” refers to botulinum toxin complexes having reduced amounts of non-toxin protein compared to the amounts naturally found in botulinum toxin complexes produced by C. botulinum bacteria. Reduced botulinum toxin complexes may be prepared using any conventional protein separation method to extract a fraction of the hemagglutinin protein or non-toxin non-hemagglutinin protein from botulinum toxin complexes derived from the bacteria. In preferred embodiments the 150 kD form is essentially free of non-toxin protein. For example, reduced botulinum toxin complexes may be produced by dissociating botulinum toxin complexes through exposure to red blood cells at a pH of 7.3, HPLC, dialysis, columns, centrifugation, and other methods for extracting proteins from complexes. Other procedures that can be used are described in, e.g., US Pat. No. 9,469,849 to Ruegg, entitled “Methods And Systems For Purifying Non-Complexed Botulinum Neurotoxin;” WO 2006/096163 to Allergan, Inc., entitled “Animal Product Free System And Process For Purifying A Botulinum Toxin;” EP 1514556 B1, to Allergan, Inc., entitled “Botulinum toxin pharmaceutical compositions,” and US 9956435 B2, Ruegg et al., “Injectable Botulinum Toxin Formulation,” each hereby incorporated herein by reference in its entirety. Alternatively, when the reduced botulinum toxin complexes are to be produced by combining synthetically produced botulinum toxin with non-toxin proteins, a reduced botulinum toxin complex is obtained by using less hemagglutinin or non-toxin, non-hemagglutinin protein in the mixture than what would be present in naturally occurring botulinum toxin complexes.

In certain exemplary embodiments, one or more non-toxin proteins are reduced by at least about 0.5%, 1%, 3%, 5%, 10%, 20%, 30%, 40%, 50%, 60%, 70%, 80%, 90% or 100% compared to the amounts normally found in botulinum toxin complexes. As noted above, Clostridium botulinum bacteria produce seven different serotypes of toxin and commercial preparations are manufactured with different relative amounts of non-toxin proteins (i.e., different amount of toxin complexes). For example, MYOBLOC™ has 5000 U of Botulinum toxin type B per ml with 0.05% human serum albumin, 0.01 M sodium succinate, and 0.1 M sodium chloride. DYSPORT™ has 500 U of botulinum toxin type A-hemagglutinin complex with 125 mcg albumin and 2.4 mg lactose. In certain embodiments, substantially all of the non-toxin protein (e.g., greater than 95%, 96%, 97%, 98% or 99% of the hemagglutinin protein and non-toxin non-hemagglutinin protein) that would normally be found in botulinum toxin complexes derived from Clostridium botulinum bacteria is removed from the botulinum toxin complex. Furthermore, although the amount endogenous non-toxin proteins may be reduced by the same amount in some cases, this invention also contemplates reducing each of the endogenous non-toxin proteins by different amounts, as well as reducing at least one of the endogenous non-toxin proteins, but not the others.

As noted above, an exogenous stabilizer (e.g., albumin) is typically added to stabilize botulinum toxin formulations. For instance, in the case of BOTOX®, 0.5 mg of human albumin per 100 U of type A botulinum toxin complex to stabilize the complex. Generally, the amount of exogenous stabilizer that may be added to stabilize the compositions according to the invention is not particularly limited. In some embodiments, the amount of added stabilizer may be less than the amount conventionally added, owing to the ability of positively charged carriers of the invention to act as a stabilizer in its own right. For instance, the amount of added exogenous albumin can be any amount less than the conventional thousand-fold excess of exogenous albumin and, in certain exemplary embodiments of the invention, is only about 0.25, 0.20, 0.15, 0.10, 0.01, 0.005, 0.001, 0.0005, 0.00001, 0.000005, 0.000001, or 0.0000001 mg per 100 U of botulinum toxin. In one embodiment, no exogenous albumin is added as a stabilizer to the compositions of the invention, thus producing albumin-free botulinum toxin compositions. Formulations that may be used with this invention that do not include albumin or other animal proteins are disclosed in U.S. Pat. Nos. 9,956,435; 10,111,939; 9,198,958; and US2009/0324647, each hereby incorporated herein by reference in its entirety.

A preferred embodiment of the invention is a liquid, botulinum toxin-containing composition that is stabilized without a proteinaceous excipient, especially without any animal protein-derived excipients. Such a liquid composition comprises a botulinum toxin, preferably botulinum toxin of serotype A, a positively charged carrier (e.g., peptide) a non-reducing disaccharide or a non-reducing trisaccharide, a non-ionic surfactant, and a physiologically compatible buffer for maintaining the pH between 4.5 and 7.5. The concentration of the non-reducing sugar in the liquid composition is in the range of 10% through 40% (w/v) and the concentration of the non-ionic surfactant is in the range of 0.005% through 0.5% (w/v). In a preferred embodiment, the botulinum toxin A has a molecular weight (MW) of 150 kDa. The preferred composition comprises botulinum toxin, preferably botulinum toxin A, more preferably, of 150 kDa MW, a positively charged carrier (e.g., peptide) as described herein, a non-reducing disaccharide, such as sucrose, a non-ionic surfactant, such as polysorbate 20, polysorbate 40, polysorbate 60, polysorbate 80, or a sorbitan ester, and a physiologically compatible buffer, such as citric acid, acetic acid, succinic acid, tartaric acid, maleic acid, and histidine; and has a pH in the range of pH 4.5. to pH 7.5. Preferred formulations are described in U.S. Pat. Nos. 9,956,435, 10,111,939, 9,198,958, and US2009/0324647, each hereby incorporated herein by reference in its entirety.

Botulinum toxin activity can be assessed using procedures known in the art, e.g., measuring units (U) of botulinum toxin activity. Median lethality assays (LD₅₀ assays) in mice are conventionally used to estimate the number of units of botulinum toxin with a high degree of precision. The amount and/or activity of the botulinum toxin in prepared formulations and compositions can be assessed using procedures practiced in the art. For example, the AlphaLISA assay, which uses two monoclonal antibodies, each specific for a distinct epitope of botulinum toxin A, may be used to quantify botulinum toxin A concentration in the ng/mL range. Alternatively or in addition, a potency test using mouse LD₅₀ may be conducted to confirm that the expected biological activity of the botulinum toxin is present. Alternatively, an HPLC assay can be used to confirm that the appropriate amount of positively charged carrier peptide is maintained in the formulation.

### Carrier Molecules

According to a preferred embodiment of the invention, a positively charged carrier molecule having protein transduction domains or efficiency groups, as described herein, has been found suitable as a transport system for a botulinum toxin, enabling toxin to be injected with improved penetration to target structures. Preferred carrier molecules are described in WO 2019/113133, entitled “Injectable botulinum toxin formulations and methods of use thereof having high response rate and long effect duration”; US 8,623,811 B2, entitled “Antimicrobial Peptide, Compositions, And Methods Of Use”; US 9,956,435 B2, entitled “Injectable Botulinum Toxin Formulations”, each hereby incorporated herein by reference in its entirety. The transport occurs without covalent modification of the botulinum toxin. Besides enhancing penetration of botulinum toxin, the positively charged carriers of the invention may, in certain preferred embodiments, stabilize the botulinum toxin against degradation. In such embodiments, the hemagglutinin protein and non-toxin, non-hemagglutinin protein that are normally present to stabilize the botulinum toxin may be reduced or omitted entirely. Similarly, the exogenous albumin that is normally added during manufacturing may be omitted.

By the use of the terms “positively charged” or “cationic” in connection with the term “carrier”, it is meant that the carrier has a positive charge under at least some solution-phase conditions, more preferably, under at least some physiologically compatible conditions. More specifically, “positively charged” and “cationic” as used herein, means that the group in question contains functionalities that are charged under all pH conditions, for instance, a quaternary amine, or contains a functionality which can acquire positive charge under certain solution-phase conditions, such as pH changes in the case of primary amines. More preferably, “positively charged” or “cationic” as used herein refers to those groups that have the behavior of associating with anions over physiologically compatible conditions. Polymers with a multiplicity of positively-charged moieties need not be homopolymers, as will be apparent to one skilled in the art. Other examples of positively charged moieties are well known in the prior art and can be employed readily, as will be apparent to those skilled in the art.

### Positively Charged Backbones of the Carrier Molecules

Generally, the positively-charged carrier (also referred to as a “positively charged backbone”) is typically a chain of atoms, either with groups in the chain carrying a positive charge at physiological pH, or with groups carrying a positive charge attached to side chains extending from the backbone. In certain preferred embodiments, the positively charged backbone is a cationic peptide. As used herein, the term “peptide” refers to an amino acid sequence, but carries no connotation with respect to the number of amino acid residues within the amino acid sequence. Accordingly, the term “peptide” may also encompass polypeptides and proteins. In certain preferred embodiments, the positively charged backbone itself will not have a defined enzymatic or therapeutic biologic activity. In certain embodiments, the backbone is a linear hydrocarbon backbone which is, in some embodiments, interrupted by heteroatoms selected from nitrogen, oxygen, sulfur, silicon and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like) but can be a heteropolymer. In one group of embodiments, the positively charged backbone is a polypropyleneamine wherein a number of the amine nitrogen atoms are present as ammonium groups (tetra-substituted) carrying a positive charge. In another embodiment, the positively charged backbone is a nonpeptidyl polymer, which may be a hetero- or homo-polymer such as a polyalkyleneimine, for example a polyethyleneimine or polypropyleneimine, having a molecular weight of from about 10,000 to about 2,500,000, preferably from about 100,000 to about 1,800,000, and most preferably from about 500,000 to about 1,400,000. In another group of embodiments, the backbone has attached a plurality of side-chain moieties that include positively charged groups (e.g., ammonium groups, pyridinium groups, phosphonium groups, sulfonium groups, guanidinium groups, or amidinium groups). The sidechain moieties in this group of embodiments can be placed at spacings along the backbone that are consistent in separations or variable. Additionally, the length of the sidechains can be similar or dissimilar. For example, in one group of embodiments, the sidechains can be linear or branched hydrocarbon chains having from one to twenty carbon atoms and terminating at the distal end (away from the backbone) in one of the above-noted positively charged groups. The association between the positively charged carrier and the botulinum toxin is by non-covalent interaction, non-limiting examples of which include ionic interactions, hydrogen bonding, van der Waals forces, or combinations thereof.

In one group of embodiments, the positively charged backbone is a polypeptide having multiple positively charged sidechain groups (e.g., lysine, arginine, ornithine, homoarginine, and the like). Preferably, the polypeptide has a molecular weight from about 100 to about 1,500,000, more preferably from about 500 to about 1,200,000, most preferably from about 1000 to about 1,000,000. One of skill in the art will appreciate that when amino acids are used in this portion of the invention, the sidechains can have either the D- or L-form (R or S configuration) at the center of attachment. In certain preferred embodiments, the polypeptide has a molecular weight from about 500 to about 5000, more preferably from 1000 to about 4000, more preferably from 2000 to about 3000. In other preferred embodiments, the polypeptide comprises 10 to 20 amino acids, or 10, 11, 12, 13, 14, 15, 16, 17, 18, 19, or 20 amino acids, preferably polylysine.

Alternatively, the backbone may comprise amino acid analogs and/or synthetic amino acids. The backbone may also be an analog of a polypeptide such as a peptoid. See, for example, Kessler, Angew. Chem. Int. Ed. Engl. 32:543 (1993); Zuckermann et al. Chemtracts-Macromol. Chem. 4:80 (1992); and Simon et al. Proc. Nat’l. Acad. Sci. USA 89:9367 (1992)). Briefly, a peptoid is a polyglycine in which the sidechain is attached to the backbone nitrogen atoms rather than the α-carbon atoms. As above, a portion of the sidechains will typically terminate in a positively charged group to provide a positively charged backbone component. Synthesis of peptoids is described in, for example, U.S. Pat. No. 5,877,278, which is hereby incorporated by reference in its entirety. As the term is used herein, positively charged backbones that have a peptoid backbone construction are considered “non-peptide” as they are not composed of amino acids having naturally occurring sidechains at the alpha-carbon locations.

A variety of other backbones can be used employing, for example, steric or electronic mimics of polypeptides wherein the amide linkages of the peptide are replaced with surrogates such as ester linkages, thioamides (—CSNH—), reversed thioamide (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene (—CR═CH—), fluoroalkene (—CF═CH—), dimethylene (—CH₂CH₂—), thioether (—CH₂S—), hydroxyethylene (—CH(OH)CH₂—), methyleneoxy (—CH₂O—), tetrazole (CN₄), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from α-amino acids.

In each of the backbones provided above, sidechain groups can be appended that carry a positively charged group. For example, the sulfonamide-linked backbones (—SO₂NH— and —NHSO₂—) can have sidechain groups attached to the nitrogen atoms. Similarly, the hydroxyethylene (—CH(OH)CH₂—) linkage can bear a sidechain group attached to the hydroxy substituent. One of skill in the art can readily adapt the other linkage chemistries to provide positively charged sidechain groups using standard synthetic methods.

### Efficiency Groups

In one embodiment, the positively charged backbone is a polypeptide having protein transduction domains (also referred to as efficiency groups). As used herein, an efficiency group or protein transduction domain is any agent that has the effect of promoting the translocation of the positively charged backbone through a tissue or cell membrane. Non-limiting examples of protein transduction domains or efficiency groups include -(gly)ₙ₁-(arg)ₙ₂ (SEQ ID NO: 5), HIV-TAT or fragments thereof, or the protein transduction domain (PTD) of Antennapedia, or a fragment thereof, in which the subscript n1 is an integer of from 0 to 20, more preferably 0 to 8, still more preferably 2 to 5, and the subscript n2 is independently an odd integer of from about 5 to about 25, more preferably about 7 to about 17, most preferably about 7 to about 13. In some embodiments, the HIV-TAT fragment does not contain the cysteine-rich region of the HIV-TAT molecule, in order to minimize the problems associated with disulfide aggregation. Preferably, the fragments of the HIV-TAT and Antennapedia protein transduction domains retain the protein transduction activity of the full protein. Still further preferred are those embodiments in which the HIV-TAT fragment has the amino acid sequence (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3) wherein the subscripts p and q are each independently an integer of from 0 to 20, or wherein p and q are each independently the integer 1. In another embodiment, the fragment or efficiency group is attached to the backbone via either the C-terminus or the N-terminus of the fragment or amino acid sequence of the efficiency group. In certain preferred embodiments, p is one and q is zero or p is zero and q is one. Preferred HIV-TAT fragments are those in which the subscripts p and q are each independently integers of from 0 to 8, more preferably 0 to 5. In another preferred embodiment the positively charged side chain or branching group is the Antennapedia (Antp) protein transduction domain (PTD), or a fragment thereof that retains activity. These are known in the art, for instance, from Console et al., J. Biol. Chem. 278:35109 (2003) and a non-limiting example of an Antennapedia PTD contemplated by this invention is the PTD having the amino acid sequence SGRQIKIWFQNRRMKWKKC (SEQ ID NO: 6). In other embodiments, the positively charged carrier is a positively charged peptide having the amino acid sequence RKKRRQRRR-G-(K)₁₅-G-RKKRRQRRR (SEQ ID NO: 4); or a positively charged peptide having the amino acid sequence YGRKKRRQRRR-G-(K)₁₅-G-YGRKKRRQRRR (SEQ ID NO: 7); or a positively charged peptide having the amino acid sequences RGRDDRRQRRR-G-(K)₁₅-G-RGRDDRRQRRR (SEQ ID NO: 8) for use in the compositions and methods of the invention.

Preferably the positively charged carrier includes side-chain positively charged protein transduction domains or positively charged efficiency groups in an amount of at least about 0.01%, as a percentage of the total carrier weight, preferably from about 0.01 to about 50 weight percent, more preferably from about 0.05 to about 45 weight percent, and most preferably from about 0.1 to about 30 weight %. For positively charged protein transduction domains having the formula -(gly)ₙ₁-(arg)ₙ₂ (SEQ ID NO: 5), a preferred range is from about 0.1 to about 25%.

In another embodiment, the backbone portion is a polylysine and positively charged protein transduction domains are attached to the lysine sidechain amino groups or to the C— or N termini. In some preferred embodiments, the polylysine may have a molecular weight that is at least 100, 200, 300, 400, 500, 600, 700, 800, 900, 1000, 1500, 2000, 2500, 3000, 3500, 4000, 4500, 5000, 5500, or 6000 D, and less than about 2,000,000, 1,000,000, 500,000, 250,000, 100,000, 75,000, 50,000, and 25,000 D. Within the range of 100 to 2,000 ,000 D, it is contemplated that the lower and/or upper range may be increased or decreased, respectively, by 100, with each resulting sub-range being a specifically contemplated embodiment of the invention. In some exemplary embodiments, the polylysine has a molecular weight from about 1,000 to about 1,500 ,000 D, from about 2,000 to about 800,000 D, or from about 3,000 to about 200,000 D. In other exemplary embodiments, the polylysine has molecular weight from about 100 to about 10,000 D, from about 500 to about 5,000 D, from about 1,000 to about 4,000 D, from about 1,500 to about 3,500 D or from about 2,000 to about 3,000 D. Preferred is a polylysine polypeptide having 10 to 20 lysines (SEQ ID NO: 9), more preferably, 15 lysines. In some embodiments, the polylysine contemplated by this invention can be any of the commercially available (Sigma Chemical Company, St. Louis, Mo., USA) polylysines such as, for example, polylysine having MW>70,000, polylysine having MW of 70,000 to 150,000, polylysine having MW 150,000 to 300,000 and polylysine having MW>300,000. The selection of an appropriate polylysine will depend on the remaining components of the composition and will be sufficient to provide an overall net positive charge to the composition and provide a length that is preferably from one to four times the combined length of the negatively charged components. Preferred positively charged protein transduction domains or efficiency groups include, for example, -gly-gly-gly-arg-arg-arg-arg-arg-arg-arg (-Gly₃Arg₇ (SEQ ID NO: 10)) or HIV-TAT.

In another preferred embodiment the positively charged backbone is a polyalkyleneimine, non-limiting examples of which include polyethyleneimine, polypropyleneimine, and polybutyleneimine. In certain embodiments, the polyalkyleneimine has a molecular weight of at least 100, 200, 300, 400, 500, 600, 700, 800, 900, 1000, 1500, 2000, 2500, 3000, 3500, 4000, 4500, 5000, 5500, or 6000 D, and less than about 2,000,000, 1,000,000, 500,000, 250,000, 100,000, 75,000, 50,000, and 25,000 D. Within the range of 100 to 2,000 ,000 D, it is contemplated that the lower and/or upper range may be increased or decreased, respectively, by 100, with each resulting sub-range being a specifically contemplated embodiment of the invention.

In other embodiments of this invention, the carrier is a relatively short polylysine or polyethyleneimine (PEI) backbone (which may be linear or branched) and which has positively charged branching groups. Without wishing to be constrained by theory, it is believed that such carriers are useful for minimizing uncontrolled aggregation of the backbones and botulinum toxin in a therapeutic composition, which causes the transport efficiency to decrease dramatically. When the carrier is a relatively short linear polylysine or PEI backbone, the backbone will have a molecular weight of less than 75,000 D, more preferably less than 30,000 D, and most preferably, less than 25,000 D. When the carrier is a relatively short branched polylysine or PEI backbone, however, the backbone will have a molecular weight less than 60,000 D, more preferably less than 55,000 D, and most preferably less than 50,000 D.

In one particularly interesting embodiment, the non-native molecules are cationic peptides that have no inherent botulinum-toxin-like activity and that also contain one or more protein transduction domains as described herein. Without wishing to be bound by any particular scientific theory, it is believed that the peptides enhance tissue penetration of molecules associated in complex after injection, while enhancing stabilization of the botulinum toxin in skin and in vitro. It is believed that the enhanced tissue penetration afforded by these peptides in particular affords reduced antigenicity, a better safety profile, enhanced potency, faster onset of clinical efficacy or longer duration of clinical efficacy compared to conventional commercial botulinum toxin complexes that are bound to exogenous albumin (e.g., BOTOX® or MYOBLOC®).

In preferred embodiments, the concentration of positively charged carriers in the compositions according to the invention is sufficient to enhance the delivery of the botulinum toxin to molecular targets such as. Furthermore, without wishing to be bound by theory, it is believed that the penetration rate follows receptor-mediated kinetics, such that tissue penetration increases with increasing amounts of penetration-enhancing-molecules up to a saturation point, upon which the transport rate becomes constant. Thus, in a preferred embodiment, the amount of added penetration-enhancing-molecules is equal to the amount that maximizes penetration rate right before saturation. A useful concentration range for the positively charged carrier (or carrier peptide) in the injectable compositions of this invention is about 0.1 pg of carrier per Unit (U) of botulinum toxin (0.1 pg/U) to about 1.0 mg per Unit (mg/U) of the botulinum toxin as described herein. A useful concentration range for the positively charged carrier (or carrier peptide) in the topical compositions of the invention is about 1.0 pg/U to 0.5 mg/U of botulinum toxin (amount of carrier/U of botulinum toxin). In other embodiments, the positively charged carrier (or carrier peptide) is present in the injectable compositions of the invention in the range of, for example, 10 ng/U to 200 ng/U of botulinum toxin, or in the range of 1 ng/U to 1000 ng/U of botulinum toxin; or in the range of 0.1 ng/U to 10,000 ng/U of botulinum toxin. In some embodiments, the amount of positively charged carrier (or carrier peptide) to Units of botulinum toxin present in the compositions of the invention is, by way of nonlimiting example, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, etc. ng of carrier per Unit of botulinum toxin (ng/U). Preferably, the botulinum toxin is of serotype A, and particularly, the 150 kD form of serotype A botulinum toxin.

### Formulation and Dosage

Compositions used in this invention are preferably in a form that permits injection into the skin of subjects or patients. The term “in need” means pharmaceutical needs (e.g., treating or preventing conditions involving undesirable headaches). In preferred embodiments, the botulinum toxin compositions for use with the invention are prepared by mixing the botulinum toxin (either containing the associated non-toxin proteins or reduced associated non-toxin proteins) with the positively charged carrier, and usually with one or more additional pharmaceutically acceptable carriers or excipients. In their simplest form, they may contain an aqueous pharmaceutically acceptable diluent, such as buffered saline (e.g., phosphate buffered saline). However, the compositions may contain other ingredients typically found in injectable pharmaceutical or cosmeceutical compositions, including a dermatologically or pharmaceutically acceptable carrier, vehicle or medium that is compatible with the tissues to which it will be applied. The term “pharmaceutically acceptable,” as used herein, means that the compositions or components thereof so described are suitable for use in contact with these tissues or for use in patients in general without undue toxicity, incompatibility, instability, allergic response, and the like. As appropriate, compositions of the invention may comprise any ingredient conventionally used in the fields under consideration.

In terms of their form, compositions for use with this invention may include solutions, emulsions (including microemulsions), suspensions, gels, powders, or other typical solid or liquid compositions used for injection to muscle and other tissues where the compositions may be used. In preferred embodiments, the compositions of the invention are present in low-viscosity, sterile formulations suitable for injection with a syringe. As used herein, the terms compositions and formulations are essentially interchangeable when referring to the compositions and formulations according to the present invention. The compositions of the invention may be in the form of a lyophilized powder that is reconstituted using a pharmaceutically acceptable liquid diluent prior to injection. The compositions of the invention may contain, in addition to the botulinum toxin and positively charged carrier, other ingredients typically used in such products, such as antimicrobials, hydration agents, tissue bulking agents or tissue fillers, preservatives, emulsifiers, natural or synthetic oils, solvents, surfactants, detergents, gelling agents, antioxidants, fillers, thickeners, powders, viscosity-controlling agents and water, and optionally including anesthetics, anti-itch actives, botanical extracts, conditioning agents, minerals, polyphenols, silicones or derivatives thereof, vitamins, and phytomedicinals.

The injectable botulinum toxin compositions for use with this invention may be in the form of controlled-release or sustained-release compositions which comprise botulinum toxin and positively charged carrier encapsulated or otherwise contained within a material such that they are released within the tissue in a controlled manner over time. The composition comprising the botulinum toxin and positively charged carrier may be contained within matrixes, liposomes, vesicles, microcapsules, microspheres and the like, or within a solid particulate material, all of which is selected and/or constructed to provide release of the botulinum toxin over time. The botulinum toxin and the positively charged carrier may be encapsulated together (i.e., in the same capsule) or separately (i.e., in separate capsules).

In embodiments, compositions of the invention comprise liquid (aqueous) compositions (or formulations) comprising a botulinum toxin as described herein, a positively charged carrier (or peptide) as described herein, a non-reducing disaccharide or a non-reducing trisaccharide, a non-ionic surfactant, and a physiologically compatible buffer, which is capable of maintaining a suitable pH, such as a pH in the range of pH 4.5 to pH 7.5, or pH 4.5 to pH 6.8, or pH 4.5 to pH 6.5. It is to be understood that a suitable pH also includes the upper and lower pH values in the range, e.g., a pH of 6.5 or a pH of 7.5. The concentration of the non-reducing sugar in the liquid composition is in the range of 10% through 40% (w/v) and the concentration of the non-ionic surfactant is in the range of 0.005% through 0.5% (w/v). The liquid compositions may be dried, preferably by lyophilization, to produce stabilized solid compositions, which may thereafter be reconstituted for use, for example, using sterile saline or other known physiologically and pharmaceutically acceptable diluents, excipients, or vehicles, especially those known for use in injectable formulations. Preferably, the dried, e.g., lyophilized, solid compositions are noncrystalline and amorphous solid compositions, and may be in the form of powders, for example. Also, preferably, the compositions of the invention do not include animal protein-derived products, such as albumin.

In certain embodiments, the compositions that may be used with the invention contain a non-reducing sugar, which is preferably a disaccharide, non-limiting examples of which include trehalose, including its anhydrous and hydrated forms, or sucrose, as well as combinations thereof. In some embodiments, the hydrated form of trehalose, trehalose-dihydrate, is preferable. In other embodiments, the compositions contain a trisaccharide, a non-limiting example of which is raffinose. In general, the concentration of the non-reducing sugar, preferably a disaccharide, e.g., sucrose, in the compositions of the invention are in the range of 10% to 40% (w/v), preferably 10% to 25% (w/v), more preferably 15% to 20% (w/v). In some preferred embodiments, the concentration of the non-reducing sugar, preferably a disaccharide, e.g., sucrose, is 10%, 11%, 12%, 13%, 14%, 15%, 16%, 17%, 18%, 19% or 20% (w/v).

In general, the compositions that may be used with the invention may include any non-ionic surfactant that has the ability to stabilize botulinum toxin and that is suitable for pharmaceutical use. In some embodiments, the non-ionic surfactant is a polysorbate, such as, by way of nonlimiting example, polysorbate 20, polysorbate 40, polysorbate 60, and polysorbate 80. In other embodiments, the non-ionic surfactant is a sorbitan ester, non-limiting examples of which include SPAN® 20, SPAN® 60, SPAN® 65, and SPAN® 80. The non-ionic surfactants Triton® X-100 or NP-40 may also be used. In addition, a combination of the different non-ionic surfactants may be used. In certain preferred embodiments, the non-ionic surfactant is a polysorbate, a poloxamer and/or a sorbitan; polysorbates and sorbitans are particularly preferred. In embodiments, the non-ionic surfactant is present in the compositions of the invention in the range of 0.005% to 0.5%, or in the range of 0.01% to 0.2%, or in the range of 0.02% to 0.1% or in the range of 0.05 to 0.08%, inclusive of the upper and lower values. In addition, the compositions of the invention may contain a non-ionic surfactant in the amount of 0.01%, 0.02%, 0.03%, 0.04%, 0.05%, 0.06%, 0.07%, 0.08%, 0.09%, 0.10%, 0.11%, 0.12%, 0.13%, 0.14%, or 0.15%.

In general for the compositions that may be used with the invention, any physiologically compatible buffer capable of maintaining the pH in the above ranges is suitable for use. Non-limiting examples of such buffers include salts of citric acid, acetic acid, succinic acid, tartaric acid, maleic acid, and histidine. Non-limiting examples of suitable buffer concentrations include buffer concentrations in the range of 0.400% to 0.600%; 0.450% to 0.575%, or 0.500% to 0.565%. The compositions of the invention may also comprise a mixture of buffer salts, non-limiting examples of which include citrate/acetate, citrate/histidine, citrate/tartrate, maleate/histidine, or succinate/histidine. Accordingly, a composition of the invention which provides a durable effect after treatment by a single injection includes a botulinum toxin, such as botulinum toxin A or botulinum toxin A of 150 kDa MW, as described herein, a positively charged carrier (or peptide) as described herein, a non-reducing disaccharide, such as sucrose, a non-ionic surfactant, such as polysorbate 20, polysorbate 40, polysorbate 60, polysorbate 80, or a sorbitan ester, and a physiologically compatible buffer, such as citric acid, acetic acid, succinic acid, tartaric acid, maleic acid, and histidine, which is capable of maintaining a suitable pH, such as a pH in the range of pH 4.5 to pH 6.5 or in the range of pH 4.5 to pH 7.5, in w/v amounts as described herein.

A particular composition for use with the invention is an albumin-free, liquid (aqueous) composition which comprises a botulinum toxin, preferably botulinum toxin of serotype A, or a botulinum toxin A having a molecular weight of 150 kDa; a positively charged carrier (e.g., peptide); a non-reducing disaccharide or a non-reducing trisaccharide, preferably a disaccharide, present in a range of 10% through 40% (w/v); a non-ionic surfactant, preferably, a polysorbate or sorbitan ester, present in the range of 0.005% through 0.5% (w/v); and a physiologically compatible buffer, such as citric acid, acetic acid, succinic acid, tartaric acid, maleic acid, or histidine, present in the range of 0.400% to 0.600%; 0.450% to 0.575%, or 0.500% to 0.565%, for maintaining the pH between 4.5 and 7.5. See, U.S. Pat. Nos. 9,956,435, 10,111,939, 9,198,958, and US2009/0324647, each hereby incorporated herein by reference in its entirety.

### Toxin Formulations

In particular embodiments the botulinum toxin composition used in this invention comprises botulinum toxin component of serotype A or serotype B. In other particular embodiments, the botulinum toxin composition comprises botulinum toxin component of serotype A which has a molecular weight of 150 kDa. In some embodiments, the botulinum toxin composition is a commercially available composition, for example DAXI (Revance), BOTOX® (Allergan, Inc.), DYSPORT® (Ipsen Limited), or MYOBLOC® (Elan Pharmaceuticals).

A suitable toxin formulation for use in the invention is a liquid, botulinum toxin-containing formulation that is stabilized without a proteinaceous excipient. In preferred embodiments the formulation is stabilized without a proteinaceous excipient, especially without any animal protein-derived excipients. Such a liquid composition comprises a botulinum toxin, preferably botulinum toxin of serotype A, a positively charged carrier (e.g., peptide) a non-reducing disaccharide or a non-reducing trisaccharide, a non-ionic surfactant, and a physiologically compatible buffer for maintaining the pH between 4.5 and 7.5. Exemplary animal-free botulinum toxin formulations for use in this invention are also described in U.S. Pat. Nos. 9,956,435, 10,111,939, 9,198,958, and US2009/0324647, each hereby incorporated herein by reference in its entirety.

Another suitable toxin formulation for use in the invention is a liquid, botulinum toxin-containing formulation that is stabilized with a proteinaceous excipient. In some embodiments, the proteinaceous excipient is an animal protein-derived excipients. Specifically, in some embodiments, the animal protein-derived excipient can be human serum albumin. An albumin-containing botulinum toxin formulation is, for example, a botulinum toxin-containing formulation that contains 100 U of type A botulinum toxin with accessory proteins, 0.5 milligrams of human albumin, and 0.9 milligrams of sodium chloride (BOTOX®). Another albumin-containing botulinum toxin formulation is a botulinum toxin-containing formulation that contains 300 U or 500 U of type A botulinum toxin with accessory proteins, 0.125 milligrams of human albumin, and 2.5 milligrams of lactose (DYSPORT®). Yes another albumin-containing botulinum toxin formulation suitable for the invention is a botulinum toxin-containing formulation that contains 5000 U of type A botulinum toxin with accessory proteins, 0.05% human serum albumin, 0.01 M sodium succinate, and 0.1 M sodium chloride (MYOBLOC®). Exemplary human/animal-protein containing botulinum toxin formulations are also described in U.S. Pat. Nos. 7,491,403, the entire contents of which are incorporated herein by reference.

### Dosing

Botulinum toxin formulations according to the invention can be delivered by injection (typically using a syringe) to areas underlying the skin, or to glandular structures within the skin, in an effective amount to interrupt signal transmission by the nerve cells to the muscles. Local delivery of botulinum toxin in this manner could lead to muscle fiber paralysis by disrupting the normal functioning of the neuromuscular junction, or block the release of acetylcholine, a neurotransmitter, from the nerve cells, thereby inhibiting muscle contractility. In addition, botulinum toxin formulations delivered in this manner could exert other biological effects, for example, a potential decrease in inflammatory mediator release.

Local delivery of the botulinum toxin in this manner could afford dosage reductions, reduce toxicity and allow more precise dosage optimization for desired effects relative to injectable or implantable materials.

The term “effective amount” or “therapeutically effective amount” as used herein means an amount of a botulinum toxin as defined above that is sufficient to produce the desired therapeutic effect. Such therapeutic effects would include reductions in either frequency, duration, or severity of any type of headache, including migraine headaches. Without being bound by theory, the therapeutic effect is believed to be mediated by the botulinum toxin biological effect, for example, by interrupting signal transmission by the nerve cells to the muscles, by disrupting of normal functioning of the neuromuscular junction, or by blocking the release of acetylcholine, but that implicitly is a safe amount, i.e., one that is low enough to avoid serious side effects.

The method of this invention may comprise administering by injection an appropriate effective amount of an injectable botulinum toxin composition for application as a single-dose treatment, or may be more concentrated, either for dilution at the place of administration or for use in multiple applications and/or sequential applications over periods of time. Through the use of this invention, a botulinum toxin composition can be administered by injection to a subject for treating headaches such as migraine headaches, preferably chronic migraine headaches or episodic migraine headaches. The botulinum toxin is administered by injection to skin-associated or other target tissue structures. In general the compositions of the invention are administered by or under the direction of a physician, clinician, medical practitioner, or other health care professional.

In preferred embodiments, a toxin is injected at a location or locations where an effect associated with botulinum toxin is desired. In the treatment or prevention of headaches, the following Table 2 provides guidance as to the appropriate dosage of reconstituted botulinum toxin formulations as described above by dose ranges 1, 2 and 3 for specific injection sites (as described below):

TABLE 3

General Guideline on Dosing Ranges by Injection Site for Headache Disorders

| | | | |
|---|---|---|---|
| | Dose Range 1 | Dose Range 2 | Dose Range 3 |
| FP1, FP2, Fpz | 2-4 | 4-8 | 8-10 |
| T3, T4 | 8-15 | 15-30 | 30-40 |
| O1, O2 | 3-8 | 8-15 | 15-20 |
| T5, T6 | 3-8 | 8-15 | 15-20 |
| F3, F4, P3, P4 | 8-15 | 15-30 | 30-40 |
| F7, F8 | 2-4 | 4-8 | 8-10 |
| Trapezius | 8-15 | 15-30 | 30-40 |
| Masseter | 8-15 | 15-30 | 30-40 |
| GND1, GND2 | 2-4 | 4-8 | 8-10 |
| Total maximum dose | Per subject: 100 U to 200 U | Per subject: 200 U to 300 U | Per subject: 300 U to 450 U |

Because of its nature, the botulinum toxin preferably is administered at an amount, application rate, and frequency that will produce the desired result without producing any adverse or undesired results. In embodiments of the invention, a single treatment with an effective dose of botulinum toxin, for example, daxibotulinumtoxinA, onabotulinumtoxinA (BOTOX®), rimabotulinumtoxinB (MYOBLOC®), and abotulinumtoxinA (DYSPORT®), affords an effect of at least 2 months or greater than 2 months, namely, 2 months, 3 months, 4 months, 5 months, 6 months, or 6 to 10 months. In an embodiment, the duration of effect of the botulinum toxin following administration to, or dosing of, an individual with botulinum toxin providing a total dose of about 100 U to 450 U; or more specifically, from about 100 U to 200 U or from about 200 U to 300 U or from about 300 U to 450 U, of botulinum toxin is, for example, at least 2 months or greater than 2 months, such as 2, 3, 4, 5, 6, or 6 to 10 months, including in between.

In certain embodiments, the compositions for use with the invention, which comprise a botulinum toxin and optionally a positively charged carrier comprising a positively charged polymeric backbone with one or more covalently attached positively charged efficiency groups as described herein, are administered as a single injection to a subject or patient in need thereof in an amount or at a dose which provides about 100 U to 450 U; or more specifically, from about 100 U to 200 U or from about 200 U to 300 U or from about 300 U to 450 U, of botulinum toxin per treatment dose per subject for the treatment or prevention of headaches. In embodiments, the botulinum toxin is of serotype A, B, C, D, E, F, or G. In an embodiment, the botulinum toxin is of serotype A. In an embodiment, the serotype A botulinum toxin has a molecular weight of 150 kDa. In an embodiment, the serotype A botulinum toxin is in the form of a higher molecular weight complex as described supra. In preferred embodiments, the 150 kDa botulinum toxin or the higher molecular weight forms of the toxin are in albumin-free formulations.

In embodiments, the composition for use with the invention is administered by injection in an amount or dose that provides 2 U or at least 2 U; 5 U or at least 5 U; 10 U or at least 10 U; 20 U or at least 20 U; 30 U or at least 30 U; 40 U or at least 40 U; 50 U or at least 50 U; 60 U or at least 60 U, 70 U or at least 70 U, 80 U or at least 80 U, 90 U or at least 90 U, or 100 U or at least 100 U of botulinum toxin per injection. Amounts or doses between the foregoing amounts or doses are also contemplated, for example, 8 U or at least 8 U; 16 U or at least 16 U; 32 U or at least 32 U, 64 U or at least 64 U and the like.

This invention also contemplates the use of a variety of delivery devices for injecting botulinum toxin-containing compositions described herein across skin. Such devices may include, without limitation, a needle and syringe, or may involve more sophisticated devices capable of dispensing and monitoring the dispensing of the composition, and optionally monitoring the condition of the subject in one or more aspects (e.g., monitoring the reaction of the subject to the substances being dispensed).

In some embodiments, the compositions can be pre-formulated and/or preinstalled in a delivery device as such. This invention also contemplates some embodiments wherein the compositions are provided in a kit that stores one or more components separately from the remaining components. For example, in certain embodiments, the invention provides for a kit that separately stores botulinum toxin and the positively charged carrier for combining at or prior to the time of application. The amount of positively charged carrier or the concentration ratio of these molecules to the botulinum toxin will depend on which carrier is chosen for use in the composition in question. The appropriate amount or ratio of carrier molecule in a given case can readily be determined, for example, by conducting one or more experiments such as those described below.

The compositions of this invention are suitable for use in physiologic environments with pH ranging from about 4.5 to about 6.3, and may thus have such a pH. However, compositions having a pH ranging from about 4.5 to about 7.5 are also embraced by the invention as described herein. The compositions according to this invention may be stored either at room temperature or under refrigerated conditions.

### Novel “Follow the Electrode” Injection Paradigm

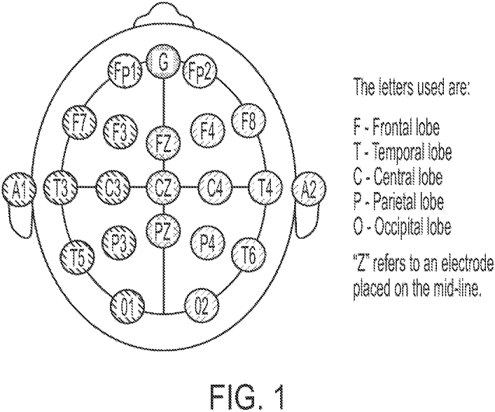

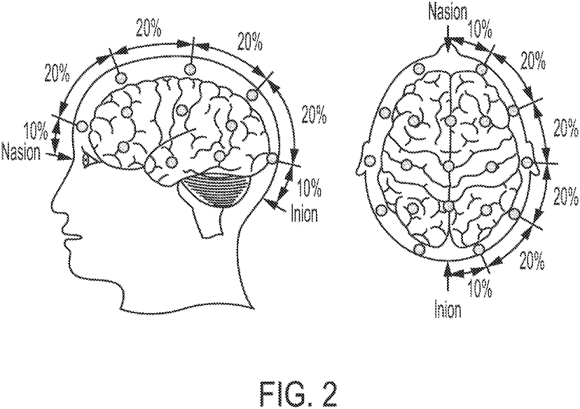

The 10/20 system is an internationally recognized method to describe the location of scalp electrodes (FIGS. 1 and 2). The system is based on the relationship between the location of an electrode and the underlying area of cerebral cortex. The number “10” and “20” refer to the fact that the distances between adjacent electrodes are either 10% or 20% of the total front-back or right-left distance of the skull (FIG. 2). Each site has a letter to identify the lobe and a number to identify the hemisphere location (Table 1). Even numbers (2, 4, 6, 8) refer to electrode positions on the right hemisphere. Odd numbers (1, 3, 5, 7) refer to electrode positions on the left hemisphere. No central lobe exists. The “C” letter is used for identification purposes only. The “z” (zero) refers to an electrode placed on the midline. Four anatomical landmarks are used for the essential positioning of the electrodes: nasion, inion, and the pre-auricular points anterior to the ear. Extra positions can be added by utilizing the spaces in between the existing 21-point 10/20 system. The 10/10 system and 10/5 system have higher density electrode settings, allowing more than 300 electrode positions. Electrode positions can be identified by using handmade position guides. Use of reference to EEG locations is only to provide reference points for injection location and is not meant to suggest actual use of EEG with electrical recordings.

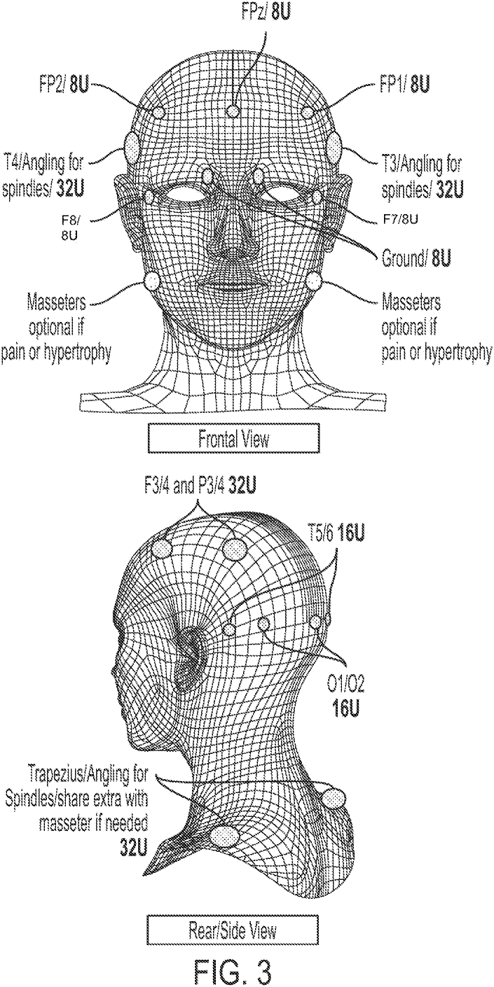

Botulinum toxin formulations for use with the invention can be delivered by subcutaneous injection (typically using a syringe) to structures underlying the skin predominantly in accordance, and in certain embodiments, in accordance with the 10/20 electrode system. As such, in some embodiments the invention focuses on delivering a therapeutically effective amount of botulinum toxin on the site of maximal benefit using a standard EEG electrode diagram as guidance (Table 1, FIGS. 1 and 2). More specifically, in some embodiments, the administration includes injection of botulinum toxin of suitable dilution using an injection paradigm that provides botulinum toxin at one or more locations of neuronal activity associated with EEG detectable brain cortical electrical activity in the individual to achieve the therapeutic effect following treatment with the botulinum toxin composition. In some embodiments, the injection sites correspond to one or more electrode placement sites selected from Fpz, Fp1, Fp2, F3, F4, F7, F8, T3, T4, C3, C4, A1, A2, T5, P3, P4, T6, O1, O2, GND1, or GND2 (Table 1). In some embodiments the injection sites further include muscle injection sites selected from trapezius muscle or masseter muscle, wherein the trapezius muscles is injected bilaterally while the masseter muscle could be injected bilaterally or unilaterally depending on if it meets clinical criteria of hypertrophy or pain. In some embodiments, the toxin is administered to the injection sites consisting of Fp1, Fp2, T3, T4, T5, T6, O1, O2, F3, F4, P3, P4, F7, F8, GND1, GND2, and Fpz. In other embodiments, the injection sites consist of Fp1, Fp2, T3, T4, T5, T6, O1, O2, F3, F4, P3, P4, F7, F8, GND1, GND2, Fpz, and trapezius muscles. In yet other embodiments, the injection sites consist of Fp1, Fp2, T3, T4, T5, T6, O1, O2, F3, F4, P3, P4, F7, F8, GND1, GND2, Fpz, right and left trapezius muscle, and right and/or left masseter muscle (FIG. 3). In some embodiments, injection sites on the patients head may be identified by using pre-made or hand-made electrode sets.

In some embodiments, the composition is administered at 10 to 25 injection sites, wherein 10 to 21 injection sites are EEG-associated injection sites and 2 to 4 injection sites are muscle injection sites; wherein the EEG-associated injection sites are selected from among Fpz, Fp1, Fp2, F3, F4, F7, F8, T3, T4, C3, C4, A1, A2, T5, P3, P4, T6, O1, O2, GND1, and GND2, and the 2 to 4 muscle injection sites are selected from right and left trapezius muscle and right and left masseter muscle. In preferred embodiments, the composition is administered at 17 injection sites; wherein the injection sites are Fp1, Fp2, T3, T4, T5, T6, O1, O2, F3, F4, P3, P4, F7, F8, GND1, GND2, and Fpz. In preferred embodiments, the composition is administered at 17 EEG-associated injection sites and 2 muscle injection sites, wherein the 17 EEG-associated injection sites are Fp1, Fp2, T3, T4, T5, T6, O1, O2, F3, F4, P3, P4, F7, F8, GND1, GND2, Fpz, and the 2 muscle sites are the right and left trapezius muscle. In yet other embodiments, the composition is administered at 17 EEG-associated injection sites and 3 muscle injection sites, wherein the 17 EEG-associated injection sites are Fp1, Fp2, T3, T4, T5, T6, O1, O2, F3, F4, P3, P4, F7, F8, GND1, GND2, Fpz, and the 3 muscle injection sites are the right and left trapezius muscle, and right or left masseter muscle. In other embodiments, the composition is administered at 17 EEG-associated injection sites and 4 muscle injection sites, wherein the 17 EEG-associated injection sites are Fp1, Fp2, T3, T4, T5, T6, O1, O2, F3, F4, P3, P4, F7, F8, GND1, GND2, Fpz, and the 4 muscle injection sites are the right and left trapezius muscle, and the right and left masseter muscle. In some embodiments, the injection sites further correspond to one or more electrode placement sites selected from Cz, Pz, or Oz. Identification of the injection location sites associated with EEG electrode placement sites, does not limit the practioner from making additional injections without departing from this invention and would be within the scope of the invention disclosed herein.

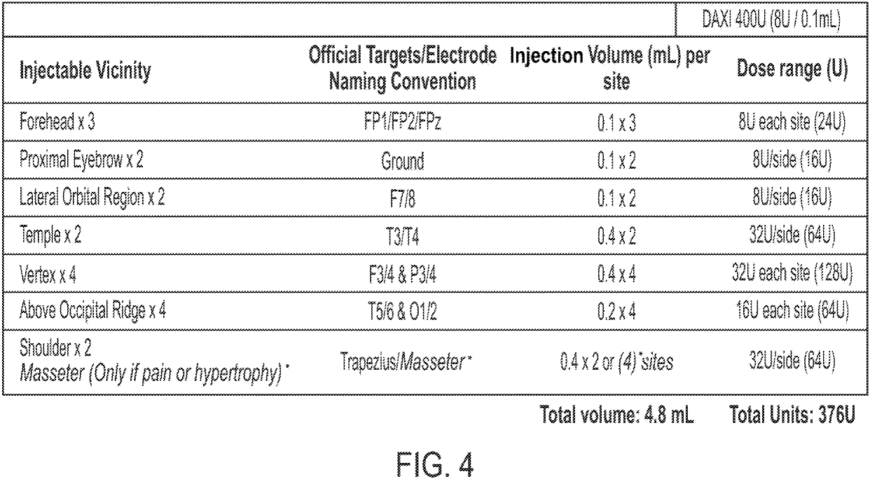

In preferred embodiments, the botulinum toxin is administered at a total dose of 376 U which is administered to a total of 19 injection sites, wherein the 17 EEG-associated injection sites are Fp1, Fp2, T3, T4, T5, T6, O1, O2, F3, F4, P3, P4, F7, F8, GND1, GND2, Fpz, and the 2 muscle injection sites are the right and left trapezius muscle; wherein 8 U are injected at injection sites Fp1, Fp2, Fpz, F7, F8, GND1, and GND2, wherein 16 U are injected at injection sites T5, T6, O1, O2, and in each trapezius muscle; and wherein 32 U are injected at injection sites T3, T4, F3, F4, P3, and P4. In embodiments, the botulinum toxin is administered at a total dose of 376 U which is administered to a total of 20 or 21 injection sites, wherein the 17 EEG-associated injection sites are Fp1, Fp2, T3, T4, T5, T6, O1, O2, F3, F4, P3, P4, F7, F8, GND1, GND2, Fpz, and the 3 or 4 muscle injection sites are the right and left trapezius muscle and right and/or left masseter muscle; wherein 8 U are injected at injection sites Fp1, Fp2, Fpz, F7, F8, GND1, and GND2, wherein 16 U are injected at injection sites T5, T6, O1, and O2, wherein 32 U are injected at injection sites T3, T4, F3, F4, P3, P4; wherein a total of 32 U which are divided based on the discretion of the injector are injected in the right trapezius muscle and right masseter muscle; and wherein a total of 32 U which are divided based on the discretion of the injector are injected in the left trapezius muscle and left masseter muscle (FIG. 4).

In preferred embodiments, the botulinum toxin is administered at a total dose of 376 U which is administered to a total of 19 injection sites, wherein the 17 EEG-associated injection sites are Fp1, Fp2, T3, T4, T5, T6, O1, O2, F3, F4, P3, P4, F7, F8, GND1, GND2, Fpz, and the 2 muscle injection sites are the right and left trapezius muscle; wherein 100 µL containing 8 U botulinum toxin are injected at injection sites Fp1, Fp2, Fpz, F7, F8, GND1, and GND2; wherein 200 µL containing 16 U botulinum toxin are injected at injection sites T5, T6, O1, O2, and in each trapezius muscle; wherein 300 µL containing 32 U botulinum toxin are injected at injection sites T3, T4, F3, F4, P3, and P4. In embodiments, the botulinum toxin is administered at a total dose of 376 U which is administered to a total of 20 or 21 injection sites, wherein the 17 EEG-associated injection sites are Fp1, Fp2, T3, T4, T5, T6, O1, O2, F3, F4, P3, P4, F7, F8, GND1, GND2, Fpz, and the 3 or 4 muscle injection sites are the right and left trapezius muscle, and right and/or left masseter muscle; wherein 100 µL containing 8 U botulinum toxin are injected at injection sites Fp1, Fp2, Fpz, F7, F8, GND1, and GND2, wherein 200 µL containing 16 U botulinum toxin are injected at injection sites T5, T6, O1, and O2, wherein 300 µL containing 32 U botulinum toxin are injected at injection sites T3, T4, F3, F4, P3, and P4; wherein 200 µL containing 16 U which are divided based on the discretion of the injector are injected in the left trapezius muscle and left masseter muscle; and wherein 200 µL containing 16 U which are divided based on the discretion of the injector are injected in the right trapezius muscle and right masseter muscle; (FIG. 4 and Table 3).

In some embodiments, the toxin of suitable dilution is administered at 10, or at least 10, or at least 11, or at least 12, or at least 13, or at least 14, or at least 15, or at least 16; 17 or at least 17; 18 or at least 18; 19 or at least 19, 20 or at least 20, 21 or at least, 22 or at least 22, 23 or at least 23, 24 or at least 24, or at least 25, in particular, 19, 20, or 21 injection sites, with the injection sites being any one of the sites listed in, but not limited to, Table 1 supra. In some embodiments, the injections site for the administration of the toxin may be selected from any of the electrode placements systems, for example, the 10/20 system, the 10/10 system, or the 10/5 system.

In embodiments, the toxin is administered by injection in an amount or dose that provides 2 U or at least 2 U per injection site; 5 U or at least 5 U per injection site; 10 U or at least 10 U per injection site; 20 U or at least 20 U per injection site; 30 U or at least 30 U per injection site; 40 U or at least 40 U per injection site; 50 U or at least 50 U per injection site; 60 U or at least 60 U per injection site, 70 U or at least 70 U per injection site, 80 U or at least 80 U per injection site, 90 U or at least 90 U per injection site, or 100 U or at least 100 U of botulinum toxin per injection. Amounts or doses between the foregoing amounts or doses are also contemplated, for example, 8 U or at least 8 U per injection site; 16 U or at least 16 U per injection site; 32 U or at least 32 U per injection site, 64 U or at least 64 U per injection site and the like.

In embodiments, the injection volume for one injection site is 100 µL or at least 100 µL per injections site; 200 µL or at least 200 µL per injection site; 300 or at least 300 µL per injection site; or 400 µL or at least 400 µL per injection site. Amounts or doses between the foregoing volumes are also contemplated, for example 50 µL, 150 µL, 250 µL, or 350 µL per injection site.

TABLE 3

Exemplary Injection Paradigm

| | | | | |
|---|---|---|---|---|
| Area | Injection Site | Injection Technique | Volume (µL) | Dose Range (U/per site) |
| Frontal Pole | FP1, FP2 | Optional: Sleeving | 100 | 8 (2 sites) |
| Frontal Pole | Fpz | | 100 | 8 (1 site) |
| Temporal Lobe | T3, T4 | Angling for muscle spindles | 400 | 32 (2 sites) |
| Temporal Lobe | T5, T6 | Sleeving downward | 200 | 16 (2 sites) |
| Occipital Lobe | O1, O2 | Sleeving downward | 200 | 16 (2 sites) |
| Frontal and Parietal Lobe | F3, F4 and P3, P4 | Optional: Sleeving | 400 | 32 (4 sites) |
| Ground | GND1, GND2 | | 100 | 8 (2 sites) |
| Frontal Lobe | F7, F8 | | 100 | 8 (2 sites) |
| Trapezius | Right, Left | Angling for muscle spindles | Total of 400 (volume per injection site based on discretion of the injector) | Total of 64 U (number of sites injected- 2, 3, or 4 -based on discretion of the injector). |
| Masseter | Right, Left | | | |

### Injection Techniques

Compositions of this invention are administered using a number of injection techniques, as described herein, allowing a tailored approach to be used when delivering the botulinum toxin to achieve an improved clinical outcome. More particularly, suitable methods for administering botulinum toxin to a subject suffering from headaches are as follows: Fanning, sleeving, angling, serial injection, bolus injection/single injection, and tunneling. The fanning technique is a method where the needle moves in multiple directions within an area and therefore there is a better chance of the toxin being distributed compared to the standard technique where the needle moves in only one direction. The product is deposited into several “pathways” from one injection site. The sleeving technique (“sleeving the needle) is a method where the full length or nearly full length of the needle is inserted to create a channel. The product is usually injected while the needle is slowly drawn backward (retrograde), so that “threads” are deposited along the length of the needle trajectory. The sleeving technique is similar to the threading injection technique used in aesthetics, but without added use of other products. The angling technique (or “angling for muscle spindles”) is a method where the needle insertion site is angled in such a way and inserted to such a depth that it gains access to muscle spindles. The single injection is a “standard” method where once the needle is inserted, it remains static during the injection. The serial injection is a puncture method where small droplets are deposited along a fine line at a short distance from one another.

In some embodiments the toxin formulation is injected using one or more injection techniques including but not limited to fanning, sleeving, tunneling, serial puncture, single puncture (“standard” or “bolus injection”), or angling. In some embodiments, the composition is injected at T5, T6, O1, and O2; wherein the injection technique used is sleeving. In some embodiments, the composition is injected at T5, T6, O1, O2, FP1, FP2, F3, F4, P3, and P4; wherein the injection technique is sleeving. In some embodiments, the composition is injected at T3, T4, and in the trapezius muscle; wherein the injection technique is angling.

The modified injection techniques described herein involve administration to allow for maximizing the dose while minimizing the number of injections required per site and thus maximizing the effect on cortical regions; while minimizing the side effects such as pain, swelling, muscle weakness, muscle paralysis, and muscle atrophy.

### Combination Therapy

In a further embodiment of the invention, the treatment or reduction in the frequency of headaches in an individual in need of treatment further comprises administering in the manner described herein a botulinum toxin composition to the individual in combination with one or more co-therapeutics. The one or more co-therapaetucs can be selected from an antagonist of calcitonin gene-related peptide (CGRP-antagonist), ergot compounds and 5-HT-1F receptor agonist. The one or more co-therapetuics can be administered seperately, sequentially, or simultaneously with the injectable botulinum toxin composition. For example, some CGRP-antagonists, including anti-CGRP antibodies can be administered by injection to a peripheral or cranial nerve, e.g., trigeminal nerves. Thus, in some aspects, the botulimin toxin composition can further comprise the CGRP-antagonist, and the combined injectable composition can be administered as a single injection at one or more injection sites.

Alternatively, the one or more co-therapuetics, such as CGRP-antagonist, can be administered seperately, sequentially, or simultaneously through other known modes of administration, e.g., orally, sublingually, transdermally, subcutaneously, intravenously, intradermally, or intramuscularly. In some embodiments. In some embodiments, the one or more co-therapeutics are administered near the end of the efficacy window of botulinum toxin, such as at about 1, 2, 3, or 4 weeks before the end of the treatment window. Efficacy window for botulinum toxin (i.e., the duratiton of action) can be from 12 weeks to 24 weeks, such as about 14 weeks, 16 weeks, 18 weeks, 20 weeks, 22 weeks, or 24 weeks. Thus, the one or more co-therapeutics can be administered at 10, 11, 12, 13, 14, 15, 16, 17, 18, 19, 20, 21, or 22 weeks after a treatment with botulinum toxin.

“Calcitonin gene-related peptide” or “CGRP,” as used herein, encompasses any member of the calcitonin family, including any calcitonin gene related peptide and analogs thereof, calcitonin, amylin, adrenomedullin, and analogs thereof. “CGRP antagonist,” as used herein, refers to any molecule that: (a) binds to CGRP or CGRP-R (CGRP receptor), and the binding results in a reduction or inhibition of CGRP activity; (b) blocks CGRP from binding to its receptor(s); (c) blocks or decreases CGRP receptor activation; (d) inhibits CGRP activity or pathways mediated by CGRP signaling function; (e) increases clearance of CGRP; or (f) inhibits or reduces CGRP synthesis, production, or release. CGRP antagonists include but are not limited to CGRP antibodies or antigen-binding fragments thereof, CGRP-R antibodies or antigen binding fragments thereof, small molecules that antagonize CGRP, and small molecules that antagonize CGRP-R.

CGRP is a 37-amino acid peptide and a potent vasodilator of almost all vascular beds. The cranial, mesenteric, and coronary arteries have been shown to be innervated with CGRP-positive fibers. CGRP has a relaxant effect on smooth muscle cells that is more prominent in the distal as opposed to the proximal coronary arteries. CGRP is also involved in nociceptive transmission in the trigeminal ganglion, the trigeminocervical complex, and the trigeminothalamic pathway. CGRP released during the activation of a migraine attack has been reported to reach plasma concentrations of ~80 pM when measured in the external jugular vein.

The CGRP receptor (CGRP-R) is a G protein-coupled receptor formed from three subunits, the receptor activity modifying protein 1 (RAMP1), a single transmembrane protein, the calcitonin-like receptor (CLR), a seven-transmembrane protein, and the intracellular receptor component protein (RCP). RAMP and CLR form the cleft for the binding of CGRP, which is the of action for gepants. RCP is responsible for intracellular signaling. RAMP1 is also a component of the amylin 1 receptor. Similarly, CLR is a component of the adrenomedullin receptor. Activation of the receptor causes cyclic adenosine monophosphate (cAMP) formation. cAMP increases protein-kinase A and can phosphorylate several downstream targets, one of which is the glutamate N-Methyl-D-aspartate (NMDA) receptor. The NMDA has a role in cortical spreading depression, which is believed to be a mechanism behind migraine aura.

In some embodiments, the CGRP-antagonist is an anti-CGRP antibody or antigen-binding fragment thereof. For example, the antibody can be galcanezumab, fremanezumab, eptinezumab, or erenumab. In some embodiments, the anti-CGRP antibody or fragment thereof can be administered at a dosage that is 20% to 80% lower than the recommended dosage for the anti-CGRP antibody monotherapy for the treatment of headache or migraine. Without wishing to be bound by theory, it is believed that combination therapy with the botulinum toxin composition permits lower dosages of the anti-CGRP antibody because CGRP can be released by peripheral nerves, and botulinum toxin is known to affect intracranial neurons implicated in disorders such as headache and migraine. Similarly, the botulinum toxin dosage, in some embodiments, may be reduced when administered in combination with an anti-CGRP antibody, e.g., reduced from 20% to 80% lower than the monotherapy dosages described herein.

In further embodiments, the CGRP-antagonist can be an intact antibody (comprising a complete or full length Fc region), a substantially intact antibody, or a portion or fragment of an antibody comprising an antigen-binding portion. Suitable antigen-binding fragments of the anti-CGRP antibody include without limitation a Fab fragment, a Fab′ fragment, or a F(ab′)2 fragment of a humanized or human antibody. Other suitable antibodies and and antigen-binding fragments include without limitation single domain antibodies, single chain variable fragments (scFv), third generation (3G) fragments, complementarity determining regions (CDRs) of scFvs, CDR-grafted antibodies, diabodies, humanized antibodies, multispecific antibodies, bispecific antibodies, DVD-Ig, chimeric antibodies and fragments thereof, camelid antibodies and fragments thereof, bovanized antibodies and fragments thereof, canine antibodies and fragments thereof, equined antibodies and fragments thereof, and felinized antibodies and fragments thereof.

Dosages and routes of administration of anti-CGRP antibodies are generally known, and include for example, parenteral, subcutaneous, or peripheral administration. Galcanezumab, for example, can be administered weekly, biweekly, monthly, every two months, every three months, every four months, every five months, or every six months at a dosage ranging from about 5 mg to about 500 mg. In some aspects, galcanezumab can be administered subcutaneously at a dose of about 10 mg to about 500 mg at time intervals ranging from every week to every ten weeks, either seperately, sequentially, or simultaneously with the administration of the botulimin toxin composition. Other galcanezumab doses that can be administered over such time intervals include from 50 mg to about 300 mg (e.g., 240 mg), 75 mg to about 250 mg (e.g., 120 mg), 75 mg to about 100 mg, and 150 mg to about 220 mg.

Similarly, erenumab can be administered weekly, biweekly, monthly, every two months, every three months, every four months, every five months or every six months at a dosage of about 5 mg to about 500 mg, either seperately, sequentially, or simultaneously with the administration of the botulimin toxin composition. In one embodiment, erenumab can be administered subcutaneously at a dose of about 5 mg to about 500 mg at intervals ranging from once per week to once every ten weeks, seperately, sequentially, or simultaneously with the administration of the botulinum toxin composition. Other erenumab doses that can be administered over such time intervals include from 10 mg to about 200 mg, 25 mg to about 150 mg (e.g., 140 mg monthly), 90 mg to about 120 mg, and 50 mg to about 60 mg.

Fremanezumab can also be administered subcutaneously, at a dose of about 100 mg to about 1000 mg every one, two, three, four, five, six, seven, eight, nine or ten weeks, either seperately, sequentially, or simultaneously with the administration of the botulinum toxin composition. Other fremanezumab doses that can be administered over such time intervals include about 150 mg to about 700 mg (e.g., 675 mg), 150 mg to about 500 mg, 150 mg to about 200 mg, and 150 mg to about 500 mg, e.g., 225 mg or 450 mg.

Eptinezumab can be administered subcutaneously at a dose of about 50 mg to about 1000 mg every one, two, three, four, five, six, seven, eight, nine or ten weeks, either seperately sequentially, or simultaneously with the administration of the botulinum toxin composition. Other eptinezumab doses that can be administered over such time intervals include about 100 mg to about 700 mg, 200 mg to about 500 mg (e.g., 200 mg), 250 mg to about 350 mg (e.g., 300 mg), and 100 mg to about 200 mg.

In further embodiments, the CGRP antagonist administered in combination with the botulinum toxin composition is a gepant. “Gepant,” as used herein, refers to a class of small molecule CGRP-antagonists, including without limitation ubrogepant, rimegepant, atogepant, and vazegepant. In general, gepants are administered orally, but other routes of administration are known and contemplated, including sublingually, transdermally, subcutaneously, intravenously, intradermally, or intramuscularly. Thus, in some embodiments, the gepant can be administered as part of the botulinum toxin composition, e.g., as a single injection at one or more injection sites.

In some embodiments, the CGRP-antagonist is ubrogepant. Ubrogepant can be administered orally, for example, at a dose of about 5 to 1000 mg once, twice, or three times per day. Other exemplary oral doses include 25 mg from 1-3 times per day, 50 mg from 1-3 times per day, or 100 mg from 1-3 times per day. In further embodiments, the CGRP antagonist is rimegepant, Similarly, in some embodiments, the CGRP antagonist can be atogepant or vazegepant. Rimegepant, atogepant, and vazegepant doses and modes of administration can be the same or similar as those for ubrogepant, e.g., from 5 mg to about 500 mg 1-3 times per day orally.

In some embodiments, a combination of the injectable botulinum toxin composition and the CGRP-antagonist can be administered to an individual that is a non-responder or insufficient responder to one or more triptan drugs. Triptan drugs include, for example, rizatriptan, sumatriptan, naratriptan, eletriptan, donitriptan, almotriptan, frovatriptan, avitriptan, and zolmitriptan. Some individuals may have a poor response with triptan drug therapy after a period of treament, typically ranging from one to twelve weeks or more. The poor response can be classified as a “non-response” where the frequency and intensity of the individuals headaches are unchanged with continued triptan therapy. An “insufficient response” is characterized by some change in frequency or intensity of headache but is nevertheless inadequate from a clinical perspective.

Some individuals with a poor response to triptan treatment may also suffer from one or more secondary headache symptoms, including for example, sinusitis, nausea, nasopharangytis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light, sounds, or smells, worsening pain during physical activity, vomiting, abdominal pain or heartburn, loss of appetite, lightheadedness, blurred vision, and fainting.

In another embodiment of the invention, the botulinum toxin composition can be administered in combination with both the CGRP-antagonist and a 5-HT-1F receptor agonist such as a ditan. 5-HT-1F receptor mRNA is found in the cortex, hippocampus, dentate gyrus, nucleus of the solitary tract, spinal cord, uterus, and mesentary. In transfected cells, the 5-HT-1F receptor is coupled to the inhibition of adenylyl cyclase. As an anti-headache or migraine drug, 5-HT-1F receptor agonist activity is associated with the inhibition of plasma extravasation in the dura, a component of neurogenic inflammation thought to be a possible cause of migraine. Activation of 5-HT-1F receptors do not mediate constriction of the human vasculature, and thus are attractive because they do not cause unwanted cardiovascular effects.

In some embodiments, the botulinum toxin composition, the CGRP-antagonist, and the 5-HT-1F receptor agonist can be administerd seperately, sequentially, or simultaneously. In some aspects, the three therapeutic agents can be administered as part of the same composition as a single injection at one or more injection sites.

Suitable 5-HT-1F receptor agonists includes a class of compounds known as ditans. A non-limiting example is lasmiditan, including pharmaceutically-acceptable salts thereof, such as the hemi-succinate salt of lasmiditan. In general, lasmiditan can be administered in doses ranging from 50 mg to about 400 mg daily dose (e.g., 100 mg or 200 mg daily dose), which can be take in aliquots at 1, 2, or 3 times per day, through various routes of administration, including orally, sublingually, transdermally, subcutaneously, intravenously, intradermally, or intramuscularly.

In another embodiment of the invention, the botulinum toxin composition can be administered in accordance with embodiments described herein in combination with both the CRGP-antagonist and an ergot compound. Ergot alkaloids and related compounds are known to have 5-HT agonist activity and have been used to treat headache such as migraine. Suitable ergot compounds including without limitation ergotamine tartrate, ergonovine maleate, and ergoloid mesylates. Other specific ergot compounds that are suitable for combination therapy include dihydroergocornine, dihydroergocristine, dihydroergocryptine, and dihydroergotamine mesylate (DHE 45). Dosing of ergot compounds is well known in the art and can be determined by a skilled clinician. The ergot compound can be administered seperately, sequentially, or simultaneously with the botulinum toxin composition and the CGRP-antagonist. In some aspects, the three therapeutic agents can be administered as part of the same composition as a single injection at one or more injection sites.

Exemplary therapeutic combinations are described in Table 4. In each of these embodiments, the botulinum toxin composition, the CGRP-antagonist, and optionally, the additional therapeutic agent(s) can be administered seperately, sequentially, or simultaneously. In some embodiments, for example, the botulinum toxin composition can be administered together as a single injection with the one or more co-therpauetics, such as CGRP-antagonist and/or the optional additional therapeutic agents. In other embodiments, the botulinum toxin composition can be administered by injection as described herein, and the one or more co-therapeutic can be administered seperately through known routes of administration (e.g., by injection for anti-CGRP antibodies and through oral dosages for small molecule gepants, 5-HT-1F receptor agonists and ergot compounds). The injectable botulinum toxin composition, in addition to the components described in the table below, can include any other suitable carriers, excipients, and the like, as described above.

TABLE 4

Exemplary Combination Therapy Embodiments

| | | |
|---|---|---|
| Botulimin Toxin Composition | CGRP-Antagonist | Optional additional therapeutic agent |
| botulinum toxin composition (100 U to 450 U or any amount therebetween) | Galcanezumab (5 mg to about 500 mg or any amount therebetween), fremanezumab (100 mg to about 1000 mg or any amount therebetween), eptinezumab (50 mg to about 1000 mg or any amount therebetween), erenumab (5 mg to about 500 mg or any amount therebetween), or combination thereof Frequency can comprise a single dose administered once every 2, 4, 8, 10, or 12 weeks. | Lasmiditan (50 mg to 400 mg daily) |
| botulinum toxin composition (100 U to 450 U or any amount therebetween) | Galcanezumab (5 mg to about 500 mg or any amount therebetween), fremanezumab (5 mg to about 1000 mg or any amount therebetween), eptinezumab (5 mg to about 1000 mg or any amount therebetween), erenumab (5 mg to about 500 mg or any amount therebetween), or combination thereof Frequency can comprise a single dose administered once every 2, 4, 8, 10, or 12 weeks. | dihydroergocomine, dihydroergocristine, dihydroergocryptine, or dihydroergotamine mesylate (DHE 45) (0.5 mg to 12 mg daily, not to exceed 10, 11, or 12 mg per week) |
| botulinum toxin composition (100 U to 450 U or any amount therebetween) | Ubrogepant (5 mg to 1000 mg daily or any amount therebetween), rimegepant (5 mg to 1000 mg daily or any amount therebetween), atogepant (5 mg to 1000 mg daily or any amount therebetween), vazegepant (5 mg to 1000 mg daily or any amount therebetween), or combination thereof Frequency of dosing can be once daily or two or more times a day as needed | Lasmiditan (50 mg to 400 mg) |
| botulinum toxin composition (100 U to 450 U or any amount therebetween) | Ubrogepant (5 mg to 1000 mg daily or any amount therebetween), rimegepant (5 mg to 1000 mg daily or any amount therebetween), atogepant (5 mg to 1000 mg daily or any amount therebetween), vazegepant (5 mg to 1000 mg daily or any amount therebetween), or combination thereof Frequency of dosing can be once daily or two or more times a day | dihydroergocomine, dihydroergocristine, dihydroergocryptine, or dihydroergotamine mesylate (DHE 45) (0.5 mg to 12 mg daily, not to exceed 10, 11, or 12 mg per week) |

In some embodiments, the botulinum toxin composition administered in combination with the one or more co-therapeutics can comprise a commercially available composition. In other ebodiments, the botulinum toxin composition can comprise the non-complexed botulinum toxin (such as the 150 kD type A botulinum toxin) and a postiviely charged carrier as described herein that is non-covalently associated wth the botulinum toxin. In some embodiments, the positively charged carrier is the positively charged backbone is polylysine or polyethyleneimine. In some embodiments, the positively charged backbone is a polylysine backbone wherein one or more positively charged efficiency groups are attached to said polylysine backbone. In some embodiments, the positively charged efficiency groups are either protected oligoarginine or TAT or modified TAT domains. In some embodiments, the positively charged efficiency groups are selected from an amino acid sequence selected from the group consisting of (gly)ₚ-RGRDDRRQRRR-(gly)_{q} (SEQ ID NO: 1), (gly)ₚ-YGRKKRRQRRR-(gly)_{q} (SEQ ID NO: 2), and (gly)ₚ-RKKRRQRRR-(gly)_{q} (SEQ ID NO: 3), wherein the subscripts p and q are each independently an integer of from 0 to 20 and mixtures thereof. In preferred embodiments, the positively charged efficiency groups include the amino acid sequence RKKRRQRRR(gly)_{q}-(K)ₓ-(gly)ₚ-RKKRRQRRR, wherein the subscript x is an integer from 5 to 50 or 10-30 or 10 to 20, wherein p and q are each an integer from 0 to 8. For instance, in a preferred embodiment, the positively charged efficiency group has the amino acid sequence RKKRRQRRRG-(K)₁₅-GRKKRRQRRR (SEQ ID NO: 4). The botulinum toxin compostion can comprise serotype A, B, C, D, E, F, G. or combinations thereof.

The dose ranges of the one or more co-therapeutics are specified in Table 4 above. For those where the dose range is 5 mg to 1000 mg, the dose can be 5 mg, 10 mg, 20 mg 30 mg, 40 mg, 50 mg, 60 mg, 70 mg, 80 mg, 90 mg, 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg. For those where the dose range is 5 mg to 500 mg, the dose can be 5 mg, 10 mg, 20 mg 30 mg, 40 mg, 50 mg, 60 mg, 70 mg, 80 mg, 90 mg, 100 mg, 125 mg, 150 mg, 175 mg, 200 mg, 250 mg, 300 mg, 350 mg, 400 mg, 450 mg, or 500 mg. For those where the dose range is 50 mg to 400 mg, the dose can be 50 mg, 60 mg, 70 mg, 80 mg, 90 mg, 100 mg, 125 mg, 150 mg, 175 mg, 200 mg, 250 mg, 300 mg, 350 mg, or 400 mg. For those where the dose range is 100 mg to 1000 mg, the dose can be 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg. For those where the dose range is 50 mg to 1000 mg, the dose can be 50 mg, 60 mg, 70 mg, 80 mg, 90 mg, 100 mg, 125 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg. For those where the dose range is 0.5 mg to 12 mg, the dose can be 0.5 mg, 1 mg, 2 mg 3 mg, 4 mg, 5 mg, 6 mg, 7 mg, 8 mg, 9 mg, 10 mg, 11 mg, or 12 mg. For localized administration, the dose may be on the low end of the range or 5%, 7%, 10%, 12%, 15%, or 20% of a systemic dose.

### EXAMPLES

### Example 1 - Use of DAXI in Chronic Migraine Using the PREEMPT Injection Paradigm

This example is an open-label, single arm, uncontrolled, multi-center clinical study of DaxibotulinumtoxinA Injectable (“DAXI”) to treat chronic migraine, a neurological condition with extremely incapacitating neurological symptoms, in adults using standard PREEMPT Injection Paradigm (PIP). Subjects with BOTOX®-responsive migraine who demonstrate the typical early wearing off from BOTOX® (efficacy < 84 days) are enrolled at multiple sites in the United States. Only subjects with headache diary reflecting both peak time of BOTOX® and wearing off are selected for this study. The cohort of subjects (18 to 64-years old) receives a total dose of up to 310 U of DAXI if using PREEMPT “fixed site” approach or a total dose of up to 390 U of DAXI if using PREEMPT “fixed site” approach plus “follow the pain” sites. All subjects are followed until they return to baseline or for up to a total of 24 weeks (168 days) after treatment. Follow up study visits are performed at 1, 2, 4, 6, and 12 weeks, and then every 4 weeks thereafter. Primary, secondary and safety endpoints are outlined in Table 5:

TABLE 5

Endpoints for Open-label Clinical Study Phase 1

| | |
|---|---|
| Endpoint | |
| Primary | Mean change in monthly migraine days after DAXI injection over successive 28 day period (for days 1-28, 29-56, 57-84) compared with BOTOX® run-in diary of same period (change from baseline) |
| Secondary | Mean change of monthly migraine days after DAXI injection over successive 28 day periods after day 84 (for days 85-112, 113-140, 114-168) or until end of study Duration of treatment effect |
| Safety | Evaluation of Adverse Events (AE) and AESI |

### Example 2 - Administering DAXI to a Subject Suffering With Headaches

This example describes administration of DaxibotulinumtoxinA Injectable (“DAXI”) to a subject suffering from chronic or episodic migraine headaches, for example high-frequency episodic migraine headaches, using an injection paradigm that provides botulinum toxin at one or more locations of neuronal activity associated with EEG detectable brain cortical electrical activity. DAXI is administered using the “follow the electrode” injection paradigm as described in Table 6.

TABLE 6

Injection Paradigm for DAXI Administration to a Subject Suffering with Headaches

| | | | | |
|---|---|---|---|---|
| Area | Injection Site | Injection Technique | Volume (µL) | Dose Range (U/per site) |
| Frontal Pole | FP1, FP2 | | 100 | 8 (2 sites) |
| Frontal Pole | Fpz | | 100 | 8 (1 site) |
| Temporal Lobe | T3, T4 | Angling for muscle spindles | 400 | 32 (2 sites) |
| Temporal Lobe | T5, T6 | Sleeving downward | 200 | 16 (2 sites) |
| Occipital Lobe | O1, O2 | Sleeving downward | 200 | 16 (2 sites) |
| Frontal and Parietal Lobe | F3, F4 and P3, P4 | | 400 | 32 (4 sites) |
| Ground | GND1, GND2 | | 100 | 8 (2 sites) |
| Frontal Lobe | F7, F8 | | 100 | 8 (2 sites) |
| Trapezius | Right, Left | Angling for muscle spindles | Total of 400 (volume per injection site based on discretion of the injector) | Total of 64 U (number of sites injected- 2, 3, or 4 -based on discretion of the injector). |
| Masseter | Right, Left | | | |

### Example 3 - Administering DAXI to a Subject Suffering With Headaches and a CGRP Antagonist, Galcanezumab,

This example describes administration of DaxibotulinumtoxinA Injectable (“DAXI”) to a subject as described n Example 2 except that the injected formulation comprises a CGRP antagonist in addition to the botulinum toxin. The dose of the CGRP antagonist in the composition would be 5-20% or 5-10% of a systemic dose for the CGRP antagonist per 300 U or per 400 U of botulinum toxin. For example, for galcanezumab, wherein a systemic dose is 120 mg, a localized dose of may be galcanezumab 6 mg to 24 mg or 6 mg to 12 mg per 300 U or per 400 U of botulinum toxin. Galcanezumab systemic dose may alternatively be 300 mg and the localzed dose would be 5-20% or 5-10% of such amount per 300 U or 400 U of botulinum toxin.

For fremanezumab and eptinezumab, a systemic dose can be an amount between 100 mg to 150 mg and the localized dose would be a percentage thereof according to the foregoing. For erenumab, , a systemic dose can be an amount between 50 mg to 200 mg and the localized dose would be a percentage thereof according to the foregoing.

1. A method of treating or reducing frequency of headaches in an individual in need of treatment, the method comprising:
administering by injection a dose of an injectable botulinum toxin composition using an injection paradigm that provides botulinum toxin at one or more locations of neuronal activity associated with EEG detectable brain cortical electrical activity in the individual to achieve the therapeutic effect following treatment with the botulinum toxin composition;
wherein the botulinum toxin composition comprises a botulinum toxin component and pharmaceutically acceptable diluent suitable for injection; ; and
wherein the total treatment dose of botulinum toxin component administered to the individual is 100 U to 450 U which is administered in one or more injections sites.

2. The method according to claim 1, wherein the headache is selected from a post-traumatic headache, a post-craniotomy headache, tension-type headache, cluster headache, and medication-overuse headache.

3. (canceled)

4. The method according to claim 2, wherein the headache is an episodic migraine headache.

5. The method according to claim 2, wherein the headache is a chronic migraine headache.

6. The method according to claim 4, wherein the episodic migraine headache is a high-frequency episodic migraine.

7. The method according to claim 1, wherein the botulinum toxin is of a serotype A.

8. The method according to claim 7, wherein the botulinum toxin is of serotype A having a molecular weight of 150 kDa.

9-17. (canceled)

18. The method according to claim 1, wherein the injection sites correspond to one or more electrode placement sites selected from Fpz, Fp1, Fp2, F3, F4, F7, F8, T3, T4, C3, C4, A1, A2, P3, P4, T5, T6, O1, O2, GND1, or GND2.

19. The method according to claim 18, wherein the injection sites further include sites selected from trapezius muscle or masseter muscle.

20. The method according to claim 19, wherein the injection sites further correspond to one or more electrode placement sites selected from Cz, Oz, or Fz.

21. The method according to claim 20, wherein the composition is administered at 10 to 25 injection sites.

22. The method according to claim 21, wherein the composition is administered to the injection sites consisting of Fp1, Fp2, T3, T4, T5, T6, O1, O2, F3, F4, P3, P4, F7, F8, GND1, GND2, and Fpz.

23. The method according to claim 21, wherein the composition is administered to the injection sites consisting of Fp1, Fp2, T3, T4, T5, T6, O1, O2, F3, F4, P3, P4, F7, F8, GND1, GND2, Fpz, and right and left trapezius muscle.

24. The method according to claim 21, wherein the composition is administered to the injection sites consisting of Fp1, Fp2, T3, T4, T5, T6, O1, O2, F3, F4, P3, P4, F7, F8, GND1, GND2, Fpz, trapezius muscles, and masseter muscles.

25. (canceled)

26. The method according to claim 22 , wherein the total treatment dose of botulinum toxin component administered to the individual is 300 U to 450 U.

27. The method according to claim 23 , wherein the total treatment dose of botulinum toxin component administered to the individual is 300 U to 450 U .

28. (canceled)

29. The method according to claim 24 , wherein the total treatment dose of botulinum toxin component to the individual is 300 U to 450 U.

30. (canceled)

31. (canceled)

32. The method for use according to claim 1 , wherein the injection volume for one injection site is from 100 to 400 µL.

33-38. (canceled)

39. The method according to claim 1, further comprising administering to the individual a co-therapeutic, wherein the co-therapeutic is a CGRP antagonist and the CGRP-antagonist is an anti-calcitonin gene-related peptide receptor antibody (anti-CGRP antibody) or an antigen-binding fragment thereof or a gepant.

40. The method according to claim 39, wherein the CGRP-antigonist is an anti-CGRP antibody selected from galcanezumab, fremanezumab, eptinezumab, erenumab, and combinations thereof.

41. The method according to claim 39, wherein the CGRP-antagonist is a gepant.

42. (canceled)

43. The method according to claim 39, wherein the CGRP-antagonist is administered sequentially, with the injectable botulinum toxin composition.

44. The method according to claim 39 , wherein the individual is a non-responder or insufficient responder to one or more triptan drugs.

45. The method according to claim 1, further comprising administering to the individual a 5-HT-1F receptor agonist.

46. The method according to claim 45, wherein the 5-HT-1F receptor agonist is a ditan or a pharmaceutically-acceptable salt thereof.

47. (canceled)

48. The method according to claim 45, wherein the injectable botulinum toxin composition, are administered separately,sequentially, or simultaneously.

49. The method according to claim 38 , further comprising administering to the individual an ergot compound.

50. (canceled)

51. (canceled)

52. The method according to claim 22, wherein the total treatment dose of botulinum toxin component administered to the individual is 200 U to 300 U.

53. The method according to claim 23, wherein the total treatment dose of botulinum toxin component administered to the individual is 200 U to 300 U.

54. The method according to claim 24, wherein the total treatment dose of botulinum toxin component administered to the individual is 200 U to 300 U.
